(19) United States
(12) Patent Application Publication
(10) Pub. No.: US 2025/0308711 A1
(43) Pub. Date: Oct. 2, 2025

(54) COLLABORATIVE FRAMEWORKS FOR IMPROVING REGULATORY DECISION-MAKING OF MEASUREMENT SOLUTIONS

(71) Applicant: Janssen Pharmaceutica NV, Beerse (BE)

(72) Inventors: Kai Langel, Beerse (BE); Bert Hartog, Beerse (BE); Erwin De beuckelaer, Beerse (BE)

(21) Appl. No.: 18/873,142

(22) PCT Filed: Jun. 9, 2023
(86) PCT No.: PCT/EP2023/065551

§ 371 Date: Dec. 9, 2024

(30) Foreign Application Priority Data
Jun. 10, 2022 (EP) 22382561.3

(51) Int. Cl. G16H 70/20; G06Q 30/018; G16H 10/20

(52) U.S. Cl. CPC G16H 70/20; G06Q 30/018; G16H 10/20

(57) ABSTRACT

In an aspect, a method of managing at least a component of a standardized digital solution for characterizing a disease is presented. The method includes obtaining, at a processor, a central data structure. The method includes receiving a request for a new standardized digital solution. The method includes generating the new standardized digital solution by leveraging one or more components of the previously approved standardized digital solutions in the central data structure. The method includes receiving feedback from a regulatory collaborator specific for a received input contributing towards at least a component of the new standardized digital solution. The method includes documenting the received feedback from the regulatory collaborator, wherein the received feedback comprises a regulatory acceptance of at least the component of the new standardized digital solution.

### CROSS-REFERENCE TO RELATED APPLICATIONS

This application claims the benefit of and priority to European Provisional Patent Application No. EP22382561.3, filed Jun. 10, 2022, and titled “Collaborative Frameworks for Improving Regulatory Decision-Making of Measurement Solutions”, the entire disclosure of which is hereby incorporated by reference in its entirety for all purposes.

### TECHNICAL FIELD

The presently disclosed subject matter generally relates to collaborative frameworks. In particular, the presently disclosed subject matter relates to collaborative frameworks for improving regulatory decision-making of measurement solutions.

### BACKGROUND

Digital health technologies show high potential in real-world evidence data generation. In the past decade, the number of clinical trials with digital health technologies involved showed a compound annual growth rate of 34.10%. However, to date, multiple limitations prevent the adoption of digital health technologies. Regularly named limitations include: (1) the lack of standardization, (2) concerns of how to choose the most appropriate digital measure, (3) how to collect, analyze and interpret the captured real-world evidence, (4) difficulty in maintaining integrity of solutions in light of everchanging technology, (5) how to prepare supporting materials for regulatory submission, and (6) the lack of translation from ideation to actual practice in clinical research and clinical care.

### SUMMARY OF THE DISCLOSURE

Disclosed herein are collaborative frameworks for achieving improved regulatory decision-making (e.g., regulatory acceptance) of harmonized, digitally-derived measures. For example, methods disclosed herein are useful for achieving improved regulatory decision-making of standardized digital solutions and/or individual components of standardized digital solutions. In various instances of the disclosure, such standardized digital solutions are valuable for characterizing diseases. Therefore, standardized digital solutions represent a uniform standard that can be provided to various parties, including regulatory parties, thereby enabling all parties to characterize diseases in a standardized fashion. Example standardized digital solutions include target solution profiles (TSPs) and digital measurement solutions (DMSs), as described in further detail herein. Additional standardized digital solutions include assets and/or components of TSPs and DMSs. Generally, methods disclosed herein involve providing a collaborative platform that brings together various stakeholders for creating standardized digital solutions.

In an aspect, a method of managing at least a component of a standardized digital solution for characterizing a disease is presented. The method includes obtaining, at a processor, a central data structure. The central data structure includes compiled evidence of a plurality of components of previously approved standardized digital solutions. The method includes receiving, at the processor, a user request for a new standardized digital solution. The method includes generating, at the processor, the new standardized digital solution by leveraging one or more components of the previously approved standardized digital solutions in the central data structure. The method includes receiving, at the processor, additional data from a user for the new standardized digital solution. The method includes updating, at the processor, the central data structure to incorporate the additional data. The method includes receiving feedback from a regulatory collaborator specific for a received input contributing towards at least a component of the new standardized digital solution. The method includes documenting the received feedback from the regulatory collaborator, wherein the received feedback comprises a regulatory acceptance of at least the component of the new standardized digital solution.

In another aspect, an apparatus for managing at least a component of a standardized digital solution for characterizing a disease is presented. The apparatus includes a processor and a memory communicatively coupled to the processor. The memory contains instructions configuring the processor to obtain a central data structure. The central data structure includes compiled evidence of a plurality of components of previously approved standardized digital solutions. The processor is configured to receive a user request for a new standardized digital solution. The processor is configured to generate a new standardized digital solution by leveraging one or more components of the previously approved standardized digital solutions in the central data structure. The processor is configured to receive additional data from a user for the new standardized digital solution. The processor is configured to update the central data structure to incorporate the additional data.

In an aspect, a method of digitally facilitating regulatory decision-making of at least a component of a standardized digital solution for characterizing a disease is presented, the method comprising: providing a digital environment comprising a plurality of components of standardized digital solutions, wherein the digital environment is accessible by a plurality of collaborators; obtaining, at a processor, a central data structure, wherein the central data structure comprises compiled evidence of a plurality of components of one or more previously-approved standardized digital solutions; receiving, at the processor, a request for a new standardized digital solution; generating, at the processor, the new standardized digital solution by leveraging one or more components of the plurality of components of the one or more previously-approved standardized digital solutions in the central data structure; receiving, at the processor, additional data for the new standardized digital solution from a collaborator of the plurality of collaborators; updating, at the processor, the central data structure to incorporate the additional data; receiving, at the processor, feedback from a regulatory collaborator, wherein the received feedback comprises a regulatory acceptance of at least a component of the new standardized digital solution; and digitally documenting, in association with the central data structure, the received feedback from the regulatory collaborator.

In an aspect, the one or more previously-approved standardized digital solutions include measurement data collected via a medical device. In various instances of the disclosure, the processor is further configured to generate a plurality of new standardized digital solutions for different patient populations. In various instances of the disclosure, the components of the one or more previously-approved standardized digital solutions are included in one or more of a validation layer, an instrumentation layer, or a definition layer of the central data structure.

In an aspect, generating the new standardized digital solution comprises analyzing evidence data of the one or more previously-approved digital solutions and generating the new standardized digital solution based on at least an acceptability categorization of the evidence data. In various instances of the disclosure, the acceptability categorization is determined according to one or more of: a freshness of the evidence data, a quantity of the evidence data, a patient population of the evidence data, regulatory acceptance of the evidence data, clinical validation of the evidence data, or technical verification of the evidence data, a content validity of the evidence data, a construct validity of the evidence data, a sensitivity to change of the evidence data, or a combination thereof.

In an aspect, generating new standardized digital solution is based on a medical condition or a diagnosis method. In various instances of the disclosure, the new standardized digital solution includes a diagnostic specific algorithm. In various instances of the disclosure, the component of the new standardized digital solution comprises any of: a measurable concept of interest component; a measurement method component; a raw data component; an algorithm component; a dataset component; a technical validation component; an analytical validation component; a clinical validation component; or a regulatory component.

In an aspect, the method further comprises, prior to receiving the collaborator request, generating a mission within a digital environment by establishing standardized roles for two or more collaborators of the plurality of collaborators, wherein each of the two or more collaborators is a discrete stakeholder in the mission. In various instances of the disclosure, establishing roles for the two or more collaborators comprises assigning a primary role to a first of the two or more collaborators and assigning a secondary role to the second of the two or more collaborators, and wherein the primary role possesses additional rights in comparison to the secondary role, wherein the rights comprise rights to access, view, and/or use results developed from the mission. In various instances of the disclosure, a collaborator of the two or more collaborators is assigned to any one of the following roles: a funder, observer, technology provider, or data partner. In various instances of the disclosure, the method further comprises, prior to receiving input from one or more of the two or more collaborators to define at least the component of the standardized digital solution, receiving agreement from each of the two or more collaborators on one or more criteria of the mission, wherein the one or more criteria of the mission comprise terms and conditions of an agreement.

In an aspect, the method further comprises, prior to receiving agreement from each of the two or more collaborators, generating a pre-defined template identifying the one or more criteria of the mission; and providing the pre-defined template to each of the two or more collaborators.

In an aspect, the new standardized digital solution is a digital measurement solution and wherein the digital measurement solution comprises a measurement definition defining one or more concepts of interest relevant to a disease; an instrumentation asset configured to transform a measurement of interest captured according to the measurement definition to a dataset that is informative for characterizing the disease, wherein the instrumentation asset of the digital measurement solution is specific for a device used to capture the measurement of interest. In various instances of the disclosure, the new standardized digital solution is a target solution profile representing a common class of digital measurement solutions; and the target solution profile comprises: a measurement definition defining one or more concepts of interest relevant to a disease; an instrumentation asset configured to transform data captured according to the measurement definition to a dataset, the instrumentation asset being device technology agnostic and is thereby interchangeable across different target solution profiles.

In an aspect, the digital environment is a collaborative platform built upon a measurement stack model.

In an aspect, the received input comprises one or more of the following types of information: technical feasibility information, patient perspective information, and clinical information.

In an aspect, the method further comprises: generating an agreement based on one or more of: the common interest, the regulator collaborator, the regulatory decision, and the established roles of the two or more collaborators, wherein the agreement comprises terms comprising one or more of: mission rules and collaborator rights; providing, via the digital environment, each of the two or more collaborators with the agreement; and providing, via the digital environment, a framework for one or more collaborators to edit the terms.

In an aspect, the method further comprises: generating a dossier for seeking regulatory decision-making, the dossier being related to at least a component of the new standardized measurement solution.

These and other aspects and features of non-limiting instances of the presently disclosed and claimed subject matter will become apparent to those skilled in the art upon review of the following description of specific non-limiting instances of the disclosed subject matter in conjunction with the accompanying drawings

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality.

FIG. 1 is a block diagram of the digital solution system, in accordance with an instance of the disclosure.

FIG. 2A is an example measurement stack, in accordance with an instance of the disclosure.

FIG. 2B is an example measurement stack showing individual components, in accordance with an instance of the disclosure.

FIG. 2C is an example measurement stack indicating one or more components that form a target solution profile (TSP), target instrumentation profile (TIP), or target component profile (TCP), in accordance with an instance of the disclosure.

FIG. 2D shows an example target solution profile, in accordance with an instance of the disclosure.

FIG. 3 is an exemplary apparatus for managing at least a component of a standardized digital solution for characterizing a disease.

FIG. 4 illustrates an exemplary acceptability criteria determination of various components of layers.

FIG. 5A depicts an exemplary target solution profile for atopic dermatitis.

FIG. 5B depicts an exemplary digital measurement solution for atopic dermatitis.

FIG. 5C depicts an example of interchangeability of assets of different digital measurement solutions for atopic dermatitis.

FIG. 6A depicts an example target solution profile for pulmonary arterial hypertension.

FIG. 6B depicts a first example digital measurement solution for pulmonary arterial hypertension.

FIG. 6C depicts a second example digital measurement solution for pulmonary arterial hypertension.

FIG. 6D depicts an example of repurposing the instrumentation asset of digital measurement solutions.

FIG. 7 depicts an example of a dynamic digital document.

FIG. 8A depicts a high level overview of exemplary collaborative efforts for developing standardized solutions.

FIG. 8B depicts an example flow process involving various parties for enabling dynamic regulatory assessment of standardized solutions.

FIG. 8C depicts an example flow process involving various parties for enabling dynamic regulatory assessment of standardized solutions.

FIG. 9 depicts a example flowchart for reusing and extending digital measurement solutions.

FIG. 10 depicts an example of a decision tree.

FIG. 11 depicts a user interface.

FIG. 12 depicts a spreadsheet showing cost savings.

FIG. 13 depicts a computing system that may be implemented in any embodiment as described throughout this disclosure.

FIG. 14 depicts a first example of an acceptability of a digital measurement solution layer.

FIG. 15 depicts a second example of an acceptability of a digital measurement solution layer.

FIG. 16 depicts a third example of an acceptability of a digital measurement solution layer.

FIG. 17 depicts a fourth example of an acceptability of a digital measurement solution layer.

### DETAILED DESCRIPTION

In the following description, for the purposes of explanation, numerous specific details are set forth in order to provide a thorough understanding of the presently disclosed subject matter. It will be apparent, however, that the presently disclosed subject matter may be practiced without these specific details. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the instances of the disclosure and are not intended to limit the scope of the disclosure, which is defined by the claims.

The term “subject” or “patient” are used interchangeably and encompass a cell, tissue, organism, human or non-human, mammal or non-mammal, male or female, whether in vivo, ex vivo, or in vitro.

The term “disease” or “condition” are used interchangeably and generally refer to a diseased status of a subject. Generally, a standardized solution, such as a digital measurement solution, is implemented to characterize the disease for the subject.

The phrase “measurement stack” refers to an organization of one or more assets that are composed of components. In particular instances of the disclosure, the measurement stack is composed of two or more assets. In particular instances of the disclosure, the measurement stack is composed of three or more assets. For example, the measurement stack includes a measurement definition asset, an instrumentation asset, and an evidence asset. The measurement stack provides a structure for standardized solutions, such as a target solution profile or a digital measurement solution.

The phrases “target solution profile” or “TSP” refer to a measurement stack in which generic descriptions are incorporated to provide a device technology agnostic profile (e.g., a profile that is independent of a particular hardware device and/or independent of particular software). In various instances of the disclosure, a target solution profile includes each of a measurement definition asset, an instrumentation asset, and an evidence asset. In various instances of the disclosure, the instrumentation asset of the target solution profile describe general methods of capturing and transforming raw data of interest, but does not specify particular devices or algorithms for capturing and transforming the raw data. Target solution profiles represent a common class of digital measurement solutions. Target solution profiles may specify performance requirements and/or standards such that digital measurement solutions of the common class represented by the target solution profile are evaluated and confirmed to perform within the performance requirements and/or standards.

The phrases “digital measurement solution” or “DMS” refer to a specific digital solution built upon a measurement stack. In various instances of the disclosure, a DMS specifies all of the components of a full solution, which can include devices, algorithms, external data, definition, and/or evidence. For example, a digital measurement solution identifies specific devices or software for capturing raw data. In various instances, a digital measurement solution identifies a specific algorithm for transforming the raw data into meaningful health data. Thus, implementation of a digital measurement solution is useful for characterizing a disease for a subject.

The phrase “standardized solution” refers to standard digital solutions useful for characterizing disease. Examples of standardized solutions include digital measurement solutions and target solution profiles.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

It must be noted that, as used in the specification, the singular forms “a,” “an” and “the” include plural referents unless the context clearly dictates otherwise.

All references, issued patents and patent applications cited within the body of the specification are hereby incorporated by reference in their entirety, for all purposes.

### Digital Solution System

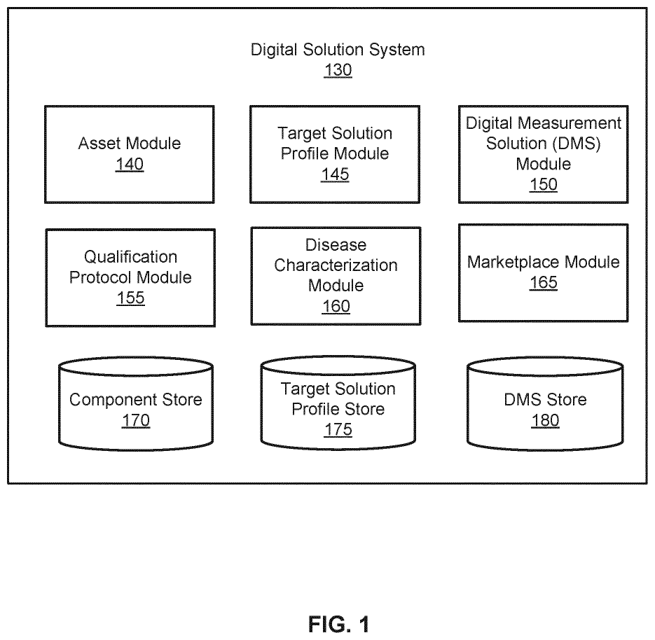

Referring now to FIG. 1, a digital solution system 130 is presented. The digital solution system includes asset module 140. The asset module 140 generates or obtains individual components and constructs assets composed of two or more components. The asset module 140 may store components and/or assets in the component store 170. Examples of components include 1) an aspect of health component relevant to the disease, 2) a hypothesis component, 3) a concept of interest component which defines a measurable unit that informs the aspect of health of the disease, 4) a measurement method component that defines how raw data is captured, 5) a raw data component specifying characteristics of the raw data, 6) an algorithm component for implementing an algorithm that transforms the raw data, 7) a health data component describing meaningful interpretation of data relevant for the disease, 8) an analytical validation component, 9) clinical validation component, and 10) a regulatory intelligence component. Further description of these example components is included herein.

In various instances, the asset module 140 may organize individual component into assets that are composed of two or more components. As an example, the asset module 140 may organize A) an aspect of health component relevant to the disease, B) a hypothesis component, and C) a concept of interest component into an asset, hereafter referred to as a measurement definition asset. As another example, the asset module 140 can organize A) a measurement method component that defines how raw data is captured, B) raw data component specifying characteristics of the raw data, C) algorithm component for implementing an algorithm that transforms the raw data, and D) health data component describing meaningful interpretation of data relevant for the disease into an asset, hereafter referred to as an instrumentation asset. As yet another example, the asset module 140 can organize A) analytical validation component, B) clinical validation component, and C) regulatory intelligence component into an asset, hereafter referred to as an evidence asset. Further details of these example assets are described herein.

In various instances, the asset module 140 generates components and constructs assets through de novo methods. For example, the asset module 140 identifies a particular disease and generates components and constructs assets that are useful for characterizing the particular disease. In various instances, the asset module 140 may receive components and/or assets from third party entities. In such instances, the third party entities may be asset developers who create and provide their own components and/or assets to the digital solution system 130. Thus, the asset module 140 can organize components received from third party entities into assets. In various instances, the asset module 140 can organize a mix of components that are generated de novo and components received from third party entities into assets.

The target solution profile module 145 generates target solution profiles (TSPs) using components and/or assets e.g., components and/or assets generated de novo by the asset module 140 or components and/or assets obtained by the asset module 140 from third party entities. In various instances, a TSP includes a measurement definition asset, instrumentation asset, and/or evidence asset. In particular instances, a TSP includes each of a measurement definition asset, instrumentation asset, and evidence asset. Generally, a TSP represents a measurement stack in which generic descriptions are incorporated to provide a device technology agnostic profile (e.g., a profile that is independent of a specific hardware device and independent of specific software). The generic descriptions are valuable to ensure that assets of the TSP can be readily interchangeable. For example, the instrumentation asset of a TSP can specify a class of devices for capturing measurements. Examples of a class of devices include, but are not limited to: wearable devices (e.g., wrist-worn device), ingestibles, image and voice based devices, touchless sensors, and sensor based smart devices (e.g., scales, thermometers, and respirators).

In various instances, the TSP module 145 builds a TSP using a condition-focused approach (e.g., bottom-up approach). Here, the TSP is built by first identifying a condition or disease of interest. Thus, the components of the TSP are assembled for the purpose of characterizing the disease of interest. In various instances, the TSP module 145 builds a TSP an instrumentation-focused approach (e.g., top-down approach). Here, the TSP is built by identifying the components and assets that are available for use (e.g., components and assets stored in component store 170). This ensures that components and assets that have previously been generated and/or validated can be easily repurposed. Thus, in various instances, building a TSP can involve repurposing components and assets from other TSPs such that new components and assets need not be generated. In particular instances, instrumentation assets of other TSPs can be repurposed for building a new TSP, even in scenarios where the other TSPs and the new TSP are developed for different diseases. The TSP module 145 can store the generated TSPs in the TSP store 175. Further details of example TSPs are described herein.

The digital measurement solution (DMS) module 150 builds one or more DMSs. In various instances, the DMS module 150 builds one or more DMSs by incorporating specific information into a TSP. Here, the TSP represents a class of solutions for the one or more DMSs. For example, the DMS module 150 can incorporate specific device hardware into a component of a TSP. Thus, a DMS specifies the particular device that is to be used to capture raw measurements. As another example, the DMS module 150 can incorporate specific algorithms into a component of a TSP. Thus, a DMS specifies the particular algorithm that is used to transform raw measurements into a meaningful health dataset that can be interpreted to characterize the disease.

In various instances, the DMS module 150 builds two or more DMSs of a common class represented by a TSP. In various instances, the DMS module 150 builds three or more DMSs, four or more DMSs, five or more DMSs, six or more DMSs, seven or more DMSs, eight or more DMSs, nine or more DMSs, ten or more DMSs, eleven or more DMSs, twelve or more DMSs, thirteen or more DMSs, fourteen or more DMSs, fifteen or more DMSs, sixteen or more DMSs, seventeen or more DMSs, eighteen or more DMSs, nineteen or more DMSs, twenty or more DMSs, twenty five or more DMSs, fifty or more DMSs, a hundred or more DMSs, two hundred or more DMSs, three hundred or more DMSs, four hundred or more DMSs, five hundred or more DMSs, six hundred or more DMSs, seven hundred or more DMSs, eight hundred or more DMSs, nine hundred or more DMSs, or a thousand or more DMSs of a common class represented by a TSP. The DMS module 150 can store the generated DMSs in the DMS store 180. Further details of example DMSs are described herein.

The qualification protocol module 155 performs qualification protocols that enable rapid onboarding of upgraded DMSs (e.g., in view of upgraded devices and/or upgraded software releases) by validating comparability of results across multiple DMSs of a common class. For example, when a new device or software package is released, the new device or software package can be incorporated in an updated or upgraded DMS. Here, the qualification protocol module 155 implements a qualification protocol to validate the new DMS incorporating the new device or new software package. This ensures that the new DMS achieves comparable results to other DMSs of the same common class.

In various instances, DMSs that have undergone successful validation using a qualification protocol can be identified as successfully validated. For example, metadata associated with a successfully validated DMS can be annotated. For example, the metadata can identify the qualification protocol that was used, as well as the fact that the DMS was successfully validated. In various instances, the metadata including the annotation can be available for inspection by a third party. Therefore, a third party, such as a customer who is interested in using a DMS to characterize a disease, can select a DMS that has been successfully validated.

The disease characterization module 160 implements a DMS to characterize a disease. In various instances, the disease characterization module 160 can be employed by a third party entity (e.g., third party entity 110 shown in FIG. 1A). For example, the third party entity may be a customer interested in characterizing a disease. Thus, the third party entity can employ the disease characterization module 160 to implement a selected DMS to characterize a disease. In various instances, the disease characterization module 160 can obtain a measurement of interest. For example, the measurement of interest can be raw data that is obtained according to the measurement method specified by the DMS. Furthermore, implementing the DMS involves transforming the measurement of interest into meaningful health data using an algorithm specified in the DMS. The disease characterization module 160 can interpret the meaningful health data to characterize the disease.

The marketplace module 165 implements a marketplace of the standardized solutions (e.g., DMSs and TSPs) and enables third party entities to access the DMSs and TSPs for their uses. In various instances, the marketplace module 165 provides an interface to third party entities that depicts the various DMSs and TSPs that are available for access. Such an interface can be organized as a catalog for ease of access.

In various instances, the marketplace module 165 provides a catalog of TSPs that are useful for characterizing various diseases. The marketplace module 165 may receive a selection of one of the TSPs. For example, a third party may select a TSP for characterizing a disease that is of interest for the third party. Furthermore, the third party may select the TSP because it includes specifications that align with the capabilities of the third party. In one scenario, the marketplace module 165 can provide the selected TSP to the third party. In one scenario, the marketplace module 165 can identify one or more DMSs that are of a common class represented by the selected TSP. Here, the marketplace module 165 provides the one or more DMSs of the common class to the third party.

In various instances, the marketplace module 165 may provide recommendations to third parties that are accessing the marketplace. For example, the marketplace module 165 can provide a recommendation identifying one or more components, one or more assets, one or more TSPs, or one or more DMSs to a third party. This can be useful for third parties that may need additional guidance as to the best standardized solution that will fit their needs.

In various instances, the marketplace module 165 receives suggestions as to additional standardized solutions that would be of value. For example, the marketplace module 165 may receive a suggestion from a third party for a particular DMS or TSP that is not present in the marketplace. Such a third party may be an asset developer or a customer who identifies a gap that is not satisfied by the current offerings of standardized solutions. For example, the suggestion may identify that specifications of a particular device exceed the specifications of available TSPs and DMSs. Therefore, the marketplace module 165 can provide the suggestion to any of the asset module 140, TSP module 145, and/or DMS module 150 to generate additional standardized solutions that can be included in the marketplace.

In various instances, the marketplace module 165 provides a catalog of target solution profiles and receives a search query. For example, a third party presented with the catalog of target solution profiles my provide a search query for a particular component or asset in a target solution profile. In various instances, the third party provides a search query for a concept of interest or for a particular disease. The marketplace module 165 queries the available TSPs (e.g., TSPs stored in the target solution profile store 175) according to the search query, and returns a list of TSPs that satisfy the search query. For example, if the search query specifies a particular concept of interest the marketplace module 165 evaluates the components of the TSPs for a concept of interest that satisfies the search query. Thus, the marketplace module 165 can provide the list of TSPs that satisfy the search query (e.g., to the third party).

Instances disclosed herein involve the building of TSPs and DMSs, as well as the implementation of TSPs and DMSs for characterizing disease. Generally, TSPs and DMSs are built on a measurement stack comprised of one or more components (also referred to herein as layers). Namely, a measurement stack provides a structure or framework for a TSP or DMS. The components and/or assets of a measurement stack can be generated and/or maintained by the asset module 140, as described above in reference to FIG. 1B.

The goal of the measurement stack is to fulfill the earlier mentioned gaps as, for example, the lack of standardization and concerns about the collection, analysis, and interpretation of data. First, the measurement stack provides a standardized structure that represents a universal way of describing a solution, thereby allowing for standardization. Second, the measurement stack initiates and allows for harmonization between multiple assets and components. Third, the measurement stack model will enable assets to transition between diseases and use-cases, enabling component level reusability.

In various instances, the measurement stack includes one or more assets. Examples of assets include a measurement definition asset, an instrumentation asset, or an evidence asset. An asset refers to one or more components of the stack. In various instances, an asset refers to two or more components. In various instances, an asset refers to three or more components. In various instances, an asset refers to three or more components.

In various instances, the measurement definition asset includes two components. In various instances, the measurement definition asset includes three components. In various instances, the measurement definition asset includes four components. In various instances, the instrumentation asset includes two components. In various instances, the instrumentation asset includes three components. In various instances, the instrumentation asset includes four components. In various instances, the evidence asset includes two components. In various instances, the evidence asset includes three components. In various instances, the evidence asset includes four components.

In various instances, the measurement stack includes two assets. For example, the measurement stack may include a measurement definition asset related to a particular disease and an instrumentation asset that describes the capturing of data that is useful for characterizing the condition. In particular instances, the measurement stack includes three assets. For example, the measurement stack may include a measurement definition asset related to a particular disease, an instrumentation asset that describes the capturing of data that is useful for characterizing the disease, and an evidence asset for validating meaningful datasets of the disease.

In various instances, the components of an asset are connected to one another. For example, the components of an asset are configured to communicate with at least one another component of the same asset. For example, within an asset, the components are organized as layers, and therefore, a first component is configured to communicate with a second component that is adjacent to the first component. This enables the transfer of information from one component to the next component.

In various instances, a component of a first asset is connected to a component of a second asset. Thus, the component of the first asset can communicate with the component of the second asset. As an example, within a measurement stack, a first asset may be located lower in the measurement stack in relation to a second asset. Here, a component of the first asset can be connected to a component of the second asset, thereby enabling the first asset and second asset to interface with each other.

In various instances, the assets of the measurement stack are ordered as follows (from bottom to top of the stack): 1) measurement definition asset and 2) instrumentation asset. In particular instances, the assets of the measurement stack are ordered as follows (from bottom to top of the stack): 1) measurement definition asset, 2) instrumentation asset, and evidence asset.

### Example Measurement Stack

Instances disclosed herein involve the building of TSPs and DMSs, as well as the implementation of TSPs and DMSs for characterizing disease. Generally, TSPs and DMSs are built on a measurement stack comprised of one or more components (also referred to herein as layers). Namely, a measurement stack provides a structure or framework for a TSP or DMS. The components and/or assets of a measurement stack can be generated and/or maintained by the asset module 140, as described above in reference to FIG. 1B. Further details of example measurement stacks are described in International Application No. WO2022234126, filed May 6, 2022, which is hereby incorporated by reference in its entirety.

The goal of the measurement stack is to fulfill the earlier mentioned gaps as, for example, the lack of standardization and concerns about the collection, analysis, and interpretation of data. First, the measurement stack provides a standardized structure that represents a universal way of describing a solution, thereby allowing for standardization. Second, the measurement stack initiates and allows for harmonization between multiple assets and components. Third, the measurement stack model will enable assets to transition between diseases and use-cases, enabling component level reusability.

In various instances, the measurement stack includes one or more assets. Examples of assets include a measurement definition asset, an instrumentation asset, or an evidence asset. An asset refers to one or more components of the stack. In various instances, an asset refers to two or more components. In various instances, an asset refers to three or more components. In various instances, an asset refers to three or more components.

In various instances, the measurement definition asset includes two components. In various instances, the measurement definition asset includes three components. In various instances, the measurement definition asset includes four components. In various instances, the instrumentation asset includes two components. In various instances, the instrumentation asset includes three components. In various instances, the instrumentation asset includes four components. In various instances, the evidence asset includes two components. In various instances, the evidence asset includes three components. In various instances, the evidence asset includes four components.

In various instances, the measurement stack includes two assets. For example, the measurement stack may include a measurement definition asset related to a particular disease and an instrumentation asset that describes the capturing of data that is useful for characterizing the condition. In particular instances, the measurement stack includes three assets. For example, the measurement stack may include a measurement definition asset related to a particular disease, an instrumentation asset that describes the capturing of data that is useful for characterizing the disease, and an evidence asset for validating meaningful datasets of the disease.

In various instances, the components of an asset are connected to one another. For example, the components of an asset are configured to communicate with at least one another component of the same asset. For example, within an asset, the components are organized as layers, and therefore, a first component is configured to communicate with a second component that is adjacent to the first component. This enables the transfer of information from one component to the next component.

In various instances, a component of a first asset is connected to a component of a second asset. Thus, the component of the first asset can communicate with the component of the second asset. As an example, within a measurement stack, a first asset may be located lower in the measurement stack in relation to a second asset. Here, a component of the first asset can be connected to a component of the second asset, thereby enabling the first asset and second asset to interface with each other.

In various instances, the assets of the measurement stack are ordered as follows (from bottom to top of the stack): 1) measurement definition asset and 2) instrumentation asset. In particular instances, the assets of the measurement stack are ordered as follows (from bottom to top of the stack): 1) measurement definition asset, 2) instrumentation asset, and evidence asset.

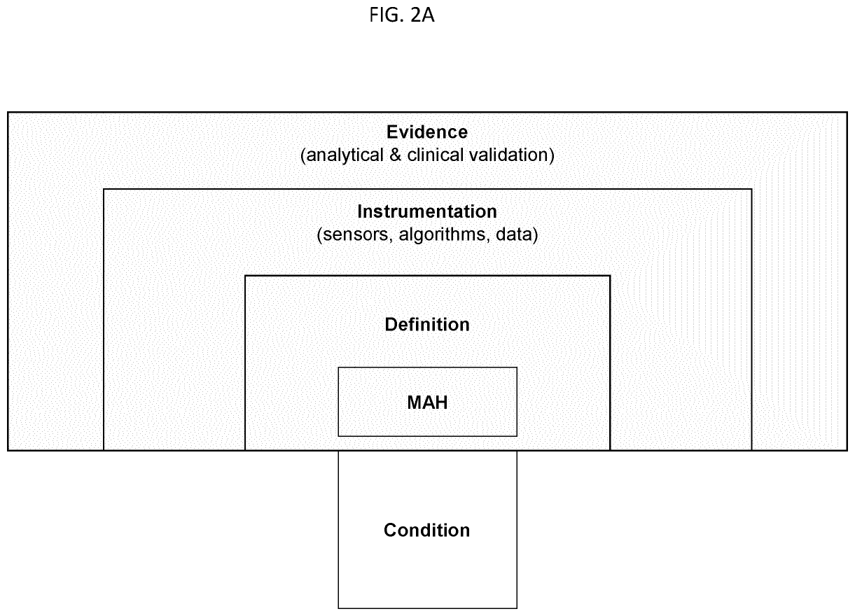

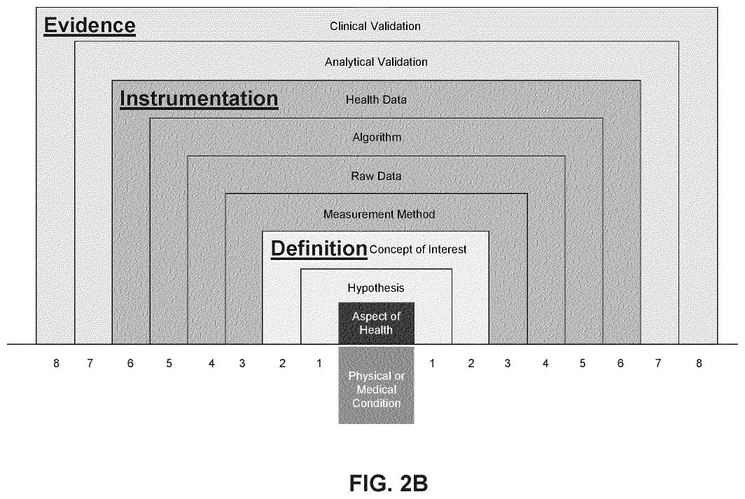

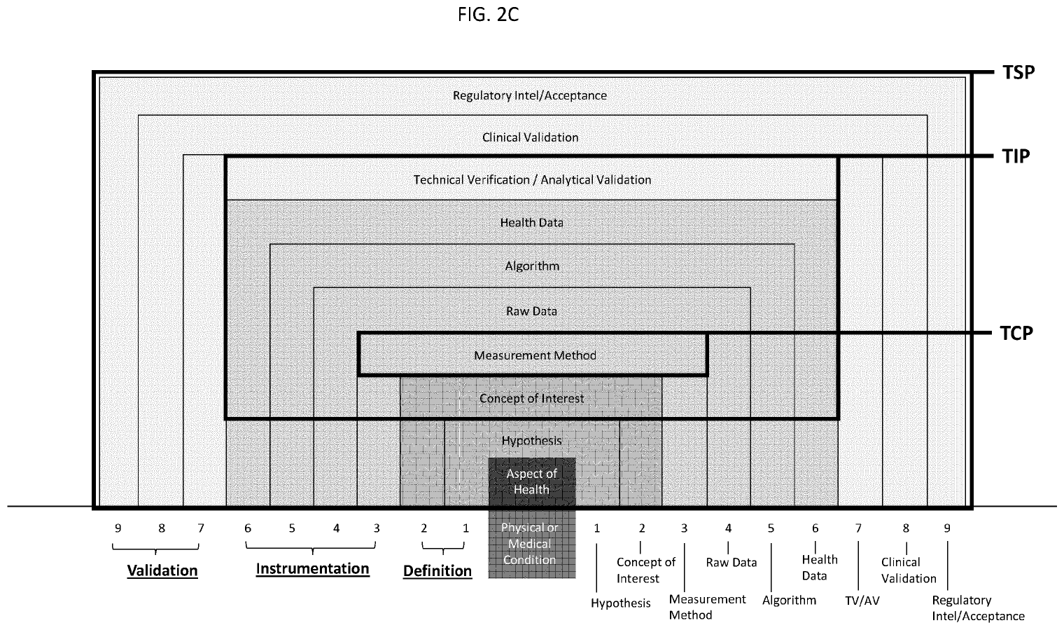

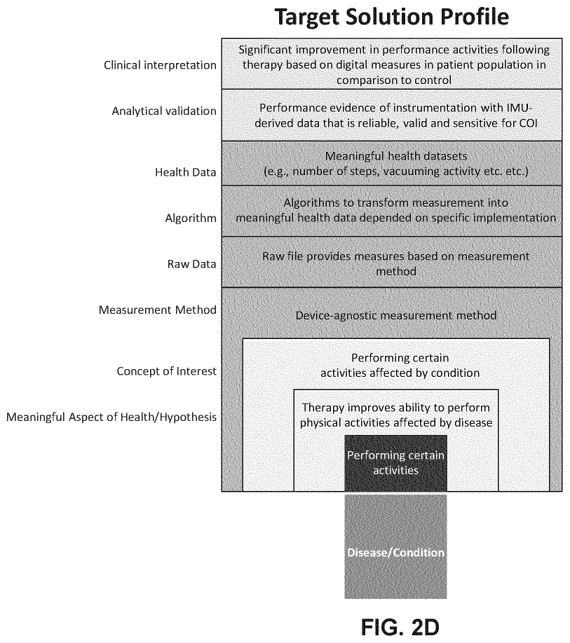

Reference is now made to FIG. 2A, which shows an example measurement stack, in accordance with an instance of the disclosure. As shown in FIG. 2A, the example measurement stack includes a particular disease (referred to as “Condition” in FIG. 2A), a measurement definition asset (labeled as “Definition” in FIG. 2A) that includes a meaningful aspect of health (MAH), an instrumentation asset (labeled as “Instrumentation” in FIG. 2A) which includes components related to the capturing of data, algorithms, and datasets, and an evidence asset (labeled as “Evidence” in FIG. 2A) that describes one or more validations for validating the generated datasets. Example conditions are further detailed in Table 1. Example meaningful aspects of health (MAH) are detailed in Table 2.

As shown in FIG. 2A, the particular disease is located at the bottom of the measurement stack. Here, the disease can govern the generation or selection of one or more of the assets above in the measurement stack. For example, the disease governs the generation or selection of the measurement definition asset.

Generally, the measurement definition asset defines measurable concepts related to the disease. Thus, the measurable concept is informative for characterizing a disease (e.g., presence of a disease, severity of a disease, progression of a disease, etc.). For example, for a condition of atopic dermatitis, the measurement definition asset may define a concept related to atopic dermatitis to be nocturnal scratching. Thus, nocturnal scratching can be measured for a subject to characterize the disease for the subject (e.g., higher quantity of nocturnal scratching can be indicative of more severe atopic dermatitis as opposed to lower quantity of nocturnal scratching). Examples of individual components of the measurement definition asset are described in further detail herein.

The instrumentation asset defines how the measurement concepts related to the disease are captured, and further defines how the captured raw data is transformed into an interpretable, meaningful health dataset. For example, the instrumentation asset can describe device specifications that influence the capture of the raw data. Furthermore, the instrumentation asset can transform the raw data into the health dataset that is more meaningful for the particular disease. The meaningful health dataset can be measurements of the concept related to the disease (as described in relation to the measurement definition asset), or can be readily interpreted to obtain measurements of the concept related to the disease. Returning to the atopic dermatitis example above, the meaningful health dataset can include a measure of nocturnal scratching (e.g., scratching events per hour, scratching duration per hour, total number of scratching events). Alternatively, the meaningful health dataset can be a dataset from which the measure of nocturnal scratching (e.g., scratching events per hour, scratching duration per hour, total number of scratching events) can be readily extracted. Examples of individual components of the instrumentation asset are described in further detail herein.

The evidence asset includes one or more validations that validate the dataset generated by the instrumentation asset. This ensures that the dataset (e.g., health dataset) generated by the instrumentation asset is accurate and can be used to accurately characterize the disease. Examples of validations included in the evidence asset can include technical validations, analytical validations, and/or clinical validations. In various instances, the evidence asset includes two or more validations. In various instances, the evidence asset includes three or more validations. Generally, performing the validations of the evidence asset ensures that measurements are accurate, and therefore, can be recognized as eligible (e.g., as a standard) for clinical trial use and approval. Examples of individual components of the evidence asset are described in further detail herein.

In various instances, the different assets of the measurement stack are selected or generated specifically for the particular condition. For example, the measurement definition asset may describe concepts particularly relevant to the condition, and therefore, the measurement definition asset may be specific for the condition. In some instances, the different assets in a measurement stack are interchangeable and can be used for measurement stacks of various diseases. For example, the instrumentation asset can be interchangeable, such that the instrumentation asset can be included in a first measurement stack for a first condition, and can further be included in a second measurement stack for a second condition. As such, interchangeable or reusable assets enables the more efficient generation and building of measurement stacks.

FIG. 2B is an example measurement stack showing individual components, in accordance with an instance of the disclosure. As shown in FIG. 2B, the measurement stack includes three assets (e.g., measurement definition asset, instrumentation asset, and evidence asset). Each of the three assets is composed of two or more components. Specifically, the measurement definition asset includes: 1) aspect of health component, 2) hypothesis component, and 3) concept of interest component. The instrumentation asset includes: 1) measurement method component, 2) raw data component, 3) algorithm component, 4) and health data component. The evidence asset includes: 1) analytical validation component, and 2) clinical validation component. As shown in FIG. 2B, there are a total of 8 components in the measurement stack.

In various instances, the measurement stack can be differently arranged such that additional or fewer components are included. In various instances, although not shown, the measurement stack can further include a regulatory component, which is valuable for aligning regulatory experts with the measurement endpoints. Such a regulatory component may be included in the evidence asset. Thus, in such instances, there are a total of 9 components in the measurement stack.

In various instances, functionalities of two or more components in an asset can be combined into a single component. Thus, there may be fewer components in the measurement stack than the 8 components that are explicitly shown in FIG. 2A. For example, the hypothesis component and the concept of interest component can be combined into a single component. As another example, the aspect of health component, the hypothesis component, and the concept of interest component can be combined into a single component.

Referring first to the condition (e.g., physical or medical condition shown in FIG. 2B), it can refer to any disease. Examples of conditions or diseases include atopic dermatitis, Parkinson's Disease, Alzheimer's Disease, chronic obstructive pulmonary disease (COPD), pulmonary arterial hypertension (PAH), asthma, retinal disease, major depressive disorder (MDD), or cancer. Additional examples of conditions are described herein and are shown in Table 1.

The meaningful aspect of health (MAH) (referred to as aspect of health in FIG. 2B), generally defines an aspect of the disease for improvement. Examples of meaningful aspects of health (MAH) are shown in Table 2. The hypothesis refers to a manner of improving the meaningful aspect of health. For example, the hypothesis can involve an intervention (e.g., a therapeutic intervention, a surgical intervention, or a change in lifestyle) that is predicted to improve the meaningful aspect of health relevant to the disease. Generally, improvement of the meaningful aspect of health correlates with improvement of the disease. In various instances, for a particular disease, there may be multiple available meaningful aspects of health (e.g., multiple aspects of the disease for improvement).

The concept of interest describes a measurable unit that informs the meaningful aspect of health. For example, the concept of interest is a measurable unit that can be used to inform the meaningful aspect of health relevant to the disease, which therefore informs the severity of the disease. Examples of concepts of interest (COI) are shown in Table 3. In various instances, the concept of interest describes a medical measurement of the disease (e.g., a measurable unit that the health care community would measure for determining severity of the disease). For example, in the context of Parkinson's disease, a medical measurement of Parkinson's is tremors. Here, the quantity of tremors can be a measure of the severity of the disease. In various instances, the concept of interest describes a measurable experience of individuals suffering from the disease. Here, the measurable experience may not be the medically relevant measurement unit, but may nonetheless have significant impact on patients afflicted with the disease. In such instances, the concept of interest can be a symptom of the disease that the patient would like to modify. For example, again in the context of Parkinson's disease, a measurable experience for individuals suffering from Parkinson's may be sleep deprivation. Although sleep deprivation is not the medical measurement unit of Parkinson's Disease, it is nonetheless a measure that can be informative of the severity of the disease.

Referring next to the measurement method component, it generally describes the solutions that are implemented for capturing data of the concept of interest. For example, solutions of the measurement method component include hardware, software, or firmware solutions. Example solutions of the measurement method component include sensors, devices such as computational devices, cellular devices or wearables, as well as mobile applications. In various instances, sensors can be built into devices, such as a wearable device or a cellular device.

In various instances, the measurement method component identifies the specifications of the measurement method. For example, for a wearable device, the measurement method component identifies the operating specifications of the wearable device (e.g., frequency or a frequency range at which the device captures data (e.g., 10-100 Hz), time intervals during which the device captures data (e.g., 24 hours a day, or in response to a command), presence of one or more sensors of the wearable device that capture data, storage capacity of the wearable device, and/or estimated battery life). In various instances, the measurement method employs products that process data captured by (mobile) sensors using algorithms to generate measures of behavioural and/or physiological function. This includes novel measures and indices of characteristics for which the underlying biological processes are not yet understood. Like other digital medicine products, these may be characterized by a body of evidence to support their quality, safety, and effectiveness as indicated in their performance requirements.

Referring next to the raw data, this component represents the raw datasets which are captured according to the particular methods of the measurement method component. For example, if the measurement method component identifies a wearable device (and the corresponding specifications), the raw data represents the dataset captured by the wearable device according to the specifications. In various instances, raw data by itself does not provide for interpretable, meaningful data. As a specific example, a raw file may include data captured at 10-100 Hz accelerations. This is captured in 3D SI units (XYZ g-force) with 28 days of continuous data collection. In short, this example describes what raw data is captured (accelerations in 3D SI units), its frequency (10-100 Hz), and the amount of data that is captured (28 days).

Referring next to the algorithm, it transforms the raw data from the raw data component into meaningful datasets (e.g., meaningful health data relevant for measuring the concept of interest). Returning again to the example of atopic dermatitis, an algorithm interprets raw measurement device data captured during sleep and transforms the raw data into meaningful health data (e.g., scratching events). In some scenarios, an algorithm is specific for a particular measurement method. Therefore, a particular algorithm in the algorithm component can only translate raw dataset outcomes that are captured from a particular measurement method.

Referring next to the health data component, it includes health data, also referred to herein as meaningful health data or meaningful health dataset. The health data is transformed by the algorithm from the raw data and represents an interpretable dataset that is informative for the particular concept of interest. Returning again to the atopic dermatitis example, health data can include, or be readily interpreted to include any of total sleep time, scratching events per hour, and the total number of scratching events. Here, health data is the outcome of algorithms/other processing to convert “raw data” into its final health-related data. One example may include converting accelerometer data into number of steps. There may be intermediary stages of this, for example identifying each episode of severe symptoms during the day could be one step, then a further refinement is the calculation of average time of all of these. Both of those could be classified as health data.

Referring next to the analytical validation component, it involves validating one or more of the other components in the measurement stack. In various instances, the input to the analytical validation include the components of the measurement definition asset, and components of the instrumentation asset. The output of the analytical validation includes supporting evidence of a successful or failed validation of the corresponding solution incorporating the components of the measurement definition asset and components of the instrumentation asset. Generally, a digital measurement solution is incomplete unless the results it generates are proven to be analytically valid to support clinical interpretation. During the analytical validation, a digital measurement solution is exposed to a series of test conditions and procedural stress to generate sample data and the results are documented for statistical analysis. The results either validate or redefine the functional range outside of which the reliability of measurements may be questionable. A successful analytical validation would mean solutions that fit the profile can support precise labelling claims without unanticipated risks or consequences.

In various instances, the analytical validation component may perform an analytical validation of device specifications, algorithms, and health data output. In various instances, the analytical validation involves comparing data to an appropriate measurement standard. Example measurement standards for various diseases can be established by third parties or in the community. For example, example measurement standards for different diseases can be standards established by ICHOM Conect.

For example, analytical validation ensures that the meaningful health data meets requisite sensitivity, specificity, and/or reliability requirements. In various instances, the requisite sensitivity requirements is any of at least 50% sensitivity, at least 60% sensitivity, at least 70% sensitivity, at least 75% sensitivity, at least 80% sensitivity, at least 85% sensitivity, at least 90% sensitivity, at least 91% sensitivity, at least 92% sensitivity, at least 93% sensitivity, at least 94% sensitivity, at least 95% sensitivity, at least 96% sensitivity, at least 97% sensitivity, at least 98% sensitivity, or at least 99% sensitivity. In various instances, the requisite specificity requirements is any of at least 50% specificity, at least 60% specificity, at least 70% specificity, at least 75% specificity, at least 80% specificity, at least 85% specificity, at least 90% specificity, at least 91% specificity, at least 92% specificity, at least 93% specificity, at least 94% specificity, at least 95% specificity, at least 96% specificity, at least 97% specificity, at least 98% specificity, or at least 99% specificity. In various instances, the requisite reliability requirements is any of at least 50% reliability, at least 60% reliability, at least 70% reliability, at least 75% reliability, at least 80% reliability, at least 85% reliability, at least 90% reliability, at least 91% reliability, at least 92% reliability, at least 93% reliability, at least 94% reliability, at least 95% reliability, at least 96% reliability, at least 97% reliability, at least 98% reliability, or at least 99% reliability.

In various instances, the analytical validation enables the comparison of the digital solution offered by the measurement stack to a reference measure that is currently employed or was previously developed for characterizing the disease. For example, returning to the example of atopic dermatitis, the analytical validation can establish that the digital solution offered by the measurement stack appropriately measures nocturnal scratching according to appropriate reliability, specificity, and sensitivity requirements. Here, the digital solution can be comparable to, or better than a reference measure (e.g., infrared observation to monitor nocturnal scratching).

In various instances, the analytical validation component includes an analytical validation and additionally or alternatively includes a technical validation. In various instances, the technical validation verifies that the datasets (e.g., raw data from the raw data component and the health data from the health data component) are appropriate. As an example, the technical validation can evaluate whether the captured raw data is in accordance with firmware and/or software protocols that are specific to the device. As another example, the technical validation can evaluate where the raw data captured by the measurement method is according to the specifications identified in the measurement method. For example, the specifications can include battery life, data storage, available measure frequencies. Therefore, the technical validation determines whether the raw data captured by the measurement method aligns with the specifications. As an example, if the raw dataset indicates that the data was captured at a frequency that exceeds the specifications identified in the measurement method, the technical validation can flag the issue and the validation process fails. Alternatively, if the raw dataset indicates that the data was captured at a frequency that is within the specifications identified in the measurement method, the technical validation can be deemed a success. Further details and examples of technical validations are described in Goldsack, J. C., et al. Verification, analytical validation, and clinical validation (V3): the foundation of determining fit-for-purpose for Biometric Monitoring Technologies (BioMeTs). npj Digit. Med. 3, 55 (2020), which is hereby incorporated by reference in its entirety.

Referring next to the clinical validation component, it involves a clinical validation of the digital solution. Clinical validation is the process that evaluates whether the measurement solution acceptably identifies, measures, or predicts a meaningful clinical, biological, physical, functional state, or experience in the specified context of use. An understanding of what level of accuracy, precision, and reliability is valuable for a solution to be useful in a specific clinical research setting. Clinical validation is intended to take a measurement that has undergone verification and analytical validation steps and evaluate whether it can answer a specific clinical question. Generally, a digital measurement solution is incomplete unless the results it generates are interpretable from a clinical perspective and sufficiently relevant to the meaningful aspects of health for the disease. Here the clinical validation component provides the guidelines to clinically interpret the measurements.

For example, the clinical validation can involve analyzing whether the digital solution identifies, measures, and predicts the meaningful clinical, biological, physical, functional state, or experience relevant for the disease.

As an example of a clinical validation, it may include guidelines identifying that a temperature measurement with a delta of 0.00001% is irrelevant for clinical decision making. Furthermore, it may identify that the standard for temperature is a delta of 0.1 degrees. In various instances, clinical validation is an in vivo validation that is performed in a specific target population. Thus, clinical validation represents a check as to whether the measurement stack is valid to answer clinical questions relevant to the disease. Returning again to the example of atopic dermatitis, the clinical validation can involve assessing the treatment effects of an intervention on nocturnal scratching within a patient population. Here, the intervention is expected to reduce the quantity of nocturnal scratching. The specific procedure identified in the clinical validation can be performed during or after the clinical trial to ensure that changes in the patient population are accurately evaluated, which provides an accurate evaluation of the impact of the intervention. Further details of clinical validation is described in Goldsack, J. C., et al. Verification, analytical validation, and clinical validation (V3): the foundation of determining fit-for-purpose for Biometric Monitoring Technologies (BioMeTs). npj Digit. Med. 3, 55 (2020), which is hereby incorporated by reference in its entirety.

In various instances, the measurement stack further includes a regulatory component for aligning regulatory experts with the measurement endpoints. Generally, the regulatory component may include regulatory guidelines. In various instances, the regulatory component includes scientific advice from third parties, such as third party regulators. For example, for a “nocturnal scratch” measure, there is a need for standardizing what “nocturnal” refers to. Thus, the regulatory component can identify guidelines for defining “nocturnal” (e.g., the start time when a person tries to go to sleep), an example of which can be a measure defined as what % of time patient is aware out of their Total Sleep Opportunity (TSO) time. Additional examples of regulatory guidelines may be standard guidelines (e.g., guidelines promulgated by the Food and Drug Administration (FDA) such as FDA patient-reported outcome (PRO) guidance or FDA's Patient Focused Drug Development (PFDD) guidance series).

In various instances, the regulatory component includes guidelines that are helpful for achieving regulatory acceptance. For example, different regulatory pathways involve different requirements to achieve regulatory acceptance. In some scenarios, requests can be submitted through FDA CPIM meetings, within an IND, or through the formal qualification procedure. In particular instances, the regulatory component assesses one or more other components of the measurement stack, such as components of the measurement definition asset and/or other components of the evidence asset. Thus, the regulatory component enables the saving of resources by involving the regulators early on as the context of use (COU), digital measures (medical device, digital biomarker, clinical outcome assessment), and all validations (e.g., technical, analytical, and clinical validation) can be approved.

In various instances, the regulatory component can be made available to third parties, such as regulators, who can further collaborate on co-developing and/or proposing improvements to standardized solutions. This enables a dynamic regulatory evaluation of standardized solutions. For example, regulators can provide new evidence requests and questions in more real time. In response, new evidence, comments, and additional context can be provided to the regulators. In various instances, the dynamic regulatory evaluation involves multiple stakeholders (e.g., involving customers, asset developers, pharmaceutical companies, regulators, etc.) and therefore, the regulatory component can be made available to the multiple stakeholders to enable a collaborative approach towards achieving regulatory approval of the standardized solution. An example of dynamic regulatory evaluation is described below in Example 5.

In various instances, regulators may evaluate the standardized solutions (e.g., DMSs) for tolerance and/or bias. Here, the regulatory component can provide guidelines for understanding the size of the expected treatment effect. If the effect is massive, tolerance can be greater, if the effect is minuscule, the measure also needs to be more precise. In various instances, the regulatory component can involve regulatory advice that is given independent of the intervention (e.g., which measures are meaningful).

FIG. 2C is an example measurement stack indicating one or more components that form a target solution profile (TSP), target instrumentation profile (TIP), or target component profile (TCP), in accordance with an instance of the disclosure. In various instances, the TSP encompasses the full measurement stack (e.g., all 9 layers as shown in FIG. 2C). In contrast, TIPs and TCPs include fewer than all the components of the measurement stack. For example, TIPs include six components (from the concept of interest up to the technical/analytical validation). TCPs refer to individual components of the measurement stack.

Generally, TSPs are considered solution-agnostic (e.g., no specific brands and versions are named). TIPs are instrumentation-centered and agnostic of certain components of the measurement definition asset (e.g., condition, meaningful aspect of health, hypothesis) as well as certain components of the evidence asset (e.g., clinical validation and regulatory intelligence). In addition, TIPs are considered condition-agnostic as no components of the TIP layers are associated with a specific condition, meaningful aspect of health, or patient population. This adds new value to the available assets provided by stakeholders and fitting these TIPs. For example, TIPs can be interchangeable across different TSPs that are designed for specific conditions. Therefore, developers (e.g., developers of individual components or assets) can develop functional assets covering multiple conditions. This is in contrast to having developers develop new assets for every specific condition and study design. Novel developments of individual assets may be a waste of resources as often the desired assets might already be available as off-the-shelf solutions. Furthermore, assets included in TIPs can readily be repurposed for multiple conditions. By implementing clinical validation for a specific condition, TIPs are applicable across various conditions, thereby allowing for improved reusability and sustainability of available assets. For example, a class of actigraphy solutions is validated to measure daily life physical activity (DLPA) for a first condition of pulmonary arterial hypertension. Analytical validation has validated the ability to measure DLPA parameters by devices included in this TIP. Additionally, DLPA can also be a concept of interest in a second condition of Parkinson's Disease (PD). Therefore, the validated TIP for measuring DLPA can be repurposed for the second condition of PD without the need to re-perform the technical and analytical validation steps, as these are already included in the TIP. Additionally, TIPs provide a structure to available assets, preventing stakeholders from being overwhelmed by the countless TSPs, devices, and algorithms accessible for digital measures. As a result, solutions within one TIP can be easily compared to deliver the best fit-for-purpose solution for the novel study design. This improves the likelihood that the best instrumentation is picked for specific use cases.

As shown in FIG. 2C, TCPs define a single generic component. TCPs thus include generic descriptions of technical details of the individual components. For example, the TCP identified in FIG. 2C can include a generic description of a measurement method (e.g., 3-axis accelerometer (wrist-worn), 10-100 Hz, 18+ hours battery life, 500+MB data storage). Generally, TCPs allow for the ease of substituting assets of the same or similar TCPs into multiple TIPs and TSPs. For example, a TSP includes a measurement method with a battery life of 18+ hours (Apple Watch 6), but a battery life of at least 96 hours is required for a specific study design. Therefore, the TCP describing 18+ hours can be substituted with a different TCP with at least 96 hours of battery life. This excludes the availability of the Apple Watch 6 in the TIP/TSP, and only components meeting the requirements will be included. TCPs thus allow researchers to best fit their specific needs with more ease.

### Example Target Solution Profile

As described herein, a TSP encompasses a full measurement stack. The generation and maintenance of TSPs can be performed by the TSP module 145, as described above in FIG. 1B. Individual components of a TSP are described using generic descriptions (in contrast to DMSs which describe specific solutions), thereby providing a device technology agnostic profile. Device technology agnostic refers to both hardware agnostic (e.g., agnostic of a particular device) as well as software agnostic (e.g., agnostic as to software version, such as software for an application or software for a device). This prevents any associations with a specific brand, model, or technology and allows for smooth emulation of specific DMSs into generic TSPs. Thus, a TSP is a generic profile that defines a class of DMSs, each of which provides further specificity to the generic profile. Since TSPs are device technology agnostic, they provide novel solutions for the same or even different conditions with more ease. This allows for the improved development of future-proof and sustainable solutions. Furthermore, TSPs provide harmonization in the ecosystem and show high potential to shorten the lifecycle of solution developments as earlier generated evidence can be repurposed. Taken together, TSPs show the potential to ultimately accelerate the adoption of digital measures in clinical research.

TSPs provide a generic description that covers multiple DMSs within the same solution class. The DMSs that fit in the class represented by the TSP can be considered fit-for-purpose for the same use case. As a result of TSPs being device technology agnostic, included DMSs show improved reusability of assets. Instead of being solely purposed for one study, TSPs accelerate the repurposing of available assets and increase the value of all assets included in DMSs. Also, TSP-classes can be leveraged to compare slight differences between similar TSPs. This allows stakeholders to compare multiple TSPs (and DMSs in the class represented by a TSP) with more ease to select the best preferences for their specific use-case (e.g., costs, the weight of the device, or battery life). In various instances, a DMS can also fit the generic description of various TSPs.

Furthermore, TSPs allow for versioning (life cycle management of digital measurement solutions). In time, available devices, algorithms, and technologies evolve. In various instances, the TSPs can be edited and modified, resulting in updated versions (which can co-exist with older versions). This allows for components of the solution to be upgraded if the solution overall still meets the TSP criteria. The validation of evolving TSPs is assessed by qualification protocols (QPs). QPs validate versioning and ensure TSPs are considered future-proof. In various instances, QPs allow for the versioning of specific assets and the ability to validate comparability between multiple DMSs.

FIG. 2D shows an example target solution profile, in accordance with an instance of the disclosure. Here, the components of the TSP are described in generic terms. However, although the TSP shown in FIG. 2D identifies a generic disease or condition, in various instances, the TSP identifies a specific disease or condition.

Referring to the measurement method component, it describes a device agnostic measurement method. For example, the measurement method component can specify a particular class of devices. Examples of a class devices can include, but are not limited to: any of wearable devices, ingestibles, image and voice based devices, touchless sensors, and sensor based smart devices (e.g., scales, thermometers, and respirators). The measurement method component can further include specifications of the measurement method e.g., battery life, data storage, available measure frequencies). Thus, a developer can determine whether the TSP is appropriate for their digital solution based on the specifications of the measurement method (e.g., if the developer needs to capture data for at least 96 hours, but the TSP measurement method specifies a battery life of 18 hours, then the developer determines that a different TSP is needed).

Referring to the raw data component of the TSP, it describes the raw file that is captured according to the measurement method. For example, the raw data according to the specifications of the measurement method. Therefore, if the measurement method indicates a measurement frequency of 100 Hz, the raw data component describes a raw file that includes data captured at the 100 Hz measurement frequency. In various instances, digital measurements reported by measurement methods are derived through a data supply chain, which includes hardware, firmware, and software components. The term “raw data” is used to describe data existing in an early stage of the data supply chain. Sensor output data at the sample level (for example, a 50 Hz accelerometer signal or a 250 Hz ECG signal) would be raw data. Although signal processing methods may have been applied to this data (e.g., down sampling, filtering, interpolation, smoothing, etc.), the data are still considered “raw” because it is a direct representation of the original signal produced by the sensor.

Referring to the algorithm component of the TSP, it identifies one or more algorithms that can appropriately transform the raw data into meaningful health data. Here, the algorithm is designed according to the specific measurement method that was used to capture the raw data. As an example, if the measurement method indicates a measurement frequency of 100 Hz, then the algorithm is designed to transform the data that was specifically captured at a frequency of Hz. In various instances, the algorithm component represents a range of data manipulation processes embedded in firmware and software, including but not limited to signal processing, data compression and decompression, artificial intelligence, and machine learning. An algorithm is a calculation that transforms the data from the sensor into meaningful information. The algorithms may be part of the sensor directly, or may be operated by a party to conduct additional data science to create a derived measure.

Referring to the analytical validation component, it enables the validation of the components of the instrumentation asset (e.g., measurement method, raw data, algorithm, and health data) to ensure that the raw data and/or health data is reliable, valid, and sensitive to meet appropriate standards. In various instances, the analytical validation occurs at the intersection of engineering and clinical expertise. It involves evaluation of the processed data and requires testing with human subjects. After verified sample-level data have been generated by a measurement method, algorithms are applied to these data in order to create behaviorally or physiologically meaningful metrics. This process begins at the point at which verified output data (sample-level data), becomes the data input for algorithmic processing. Therefore, the first step of analytical validation requires a defined data capture protocol and a specified test subject population. During the process of analytical validation, the metric produced by the algorithm is evaluated against an appropriate reference standard.

In various instances, a TSP can be built using a condition-focused approach (e.g., bottom-up approach). In various instances, a TSP can be built using an instrumentation-focused approach (e.g., top-down approach). Regardless of the approach (e.g., bottom up or top down), the final TSP and DMS(s) of the class can be identical.

Referring first to the condition-focused approach, a specific condition is identified. Here, a measurement definition meaningful for patients with the condition is determined. This includes determining the concept of interest that will be measured. Next, suitable instrumentation is developed, or, if available, off-the-shelf solutions could be selected. For example, an instrumentation asset of a different TSP could be selected and repurposed for this current TSP. Given that the instrumentation asset of TSPs is generally described in generic terms, the repurposing of the instrumentation asset for the current TSP can require little or no additional work. Next, an evidence asset is generated for the TSP. In various instances, generating an evidence asset involves determining technical and analytical validations that are appropriate for the instrumentation of the TSP. In various instances, components of the evidence asset, such as components for performing technical and analytical validations, can be repurposed from another TSP. Given that technical and analytical validations may have previously been performed for a generic instrumentation asset of another TSP, the current TSP need not re-perform the same technical and analytical validations again. In various instances, a component of the evidence asset includes a clinical validation component. Here, the clinical validation component verifies whether results are clinically valid. Thus, generating an evidence asset includes generating the clinical validation component that is valuable for ensuring the results of the TSP are of clinical value.

Referring to the instrumentation-focused approach, it begins with the discovery of available components. Components with similar instrumentation are identified and thereafter profiled into classes of solutions. These individual components are profiled into individual component classes known as TCPs (solely one generically described layer of the measurement stack, e.g., measurement method or algorithm). Multiple TCPs of different layers can be united into TIPs if completed with the concept of interest, measurement method, raw data, algorithm, health data, and technical verification/analytical validation. TSPs are built on top of these TIPs with aligned definitions and validated instrumentation by completing them with the condition-related layers. For example, TSPs are built by generating the meaningful aspect of health component and clinical validation component on top of the components of the TIPs.

The instrumentation-focused approach eases the development of TCPs, multiple TIPs, and numerous TSPs. In the instrumentation-focused approach, smaller stakeholders could easier contribute to the development of assets, as they often have assets as measurement methods and algorithms within their digital portfolios. Asset providers can now focus more on the development of one component, which can be useful for multiple studies.

In various instances, given a full TSP, assets and components of the TSP can be quickly drafted. For example, TIPs and TCPs can quickly be drafted from a complete TSP by excluding the definition-related layers or by picking one individual layer, respectively.

As described herein, assets and components of TSPs can be interchangeable and substituted. For example, a TIP from a first TSP can be substituted for in place of a second TIP in a second TSP. Here, substituting a TIP can be beneficial as it minimizes the validations that are required in view of the substitution. For example, only a technical validation/analytical validation for TSP including the now substituted TSP is re-assessed. As another example, only a clinical validation for the TSP including the now substituted TSP is re-assessed (e.g., technical/analytical validation need not be performed).

### Overview of an Apparatus for Managing at Least a Component of a Standardized DMS

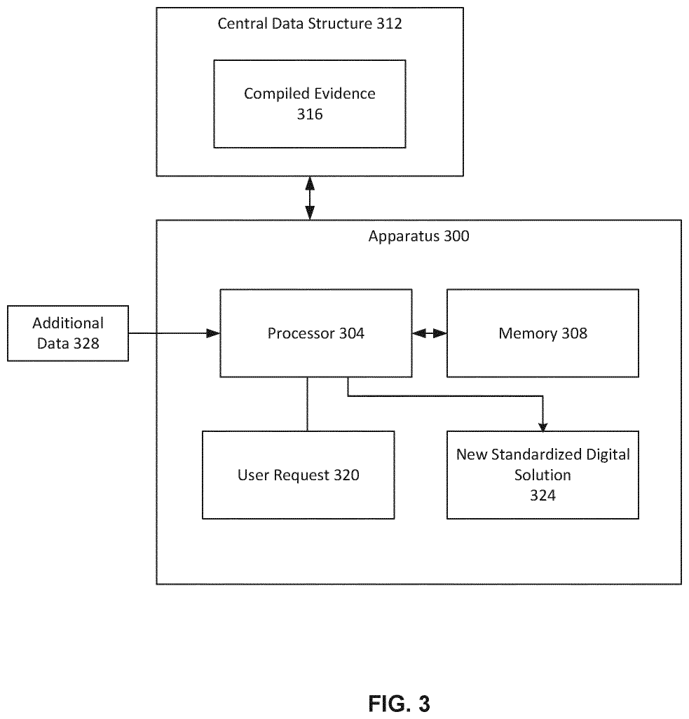

Referring now to FIG. 3, an apparatus 300 for managing at least a component of a standardized digital solution for characterizing a disease is presented. Apparatus 300 may include a processor 304 and a memory coupled and/or in communication with the processor 304. The memory may contain instructions configuring the processor perform various tasks. In some instances, the processor 304 may be configured to obtain central data structure 312. The central data structure 312 may be obtained over a network connection, may be retrieved from the memory 308, and/or may be communicated with one or more external computing devices.

The central data structure 312 may include one or more databases. In some instances, the central data structure 312 may include compiled evidence 316 of a plurality of components of previously approved standardized digital solutions. Standardized digital solutions may include digital measurement solutions, targets solution profiles, and/or components thereof. For instance, compiled evidence 316 may include a standardized digital solution in a form of a DMS. The compiled evidence 316 may include data of one or more components of a DMS, such as, without limitation, raw sensor data, hardware data, software data, diagnostic data, algorithmic data, disease data, patient data, and/or other types of data of one or more components of the DMS. In some instances, the compiled evidence 316 may include a standardized digital solution in a form of a target solution profile. For instance, the compiled evidence data 316 may include data of one or more components of a TSP, such as, without limitation, measurement data, instrument data, data of methods of capturing and transforming raw data, common class data of one or more DMS, and the like. In some instances, the compiled evidence 316 may be previously approved by one or more individuals, organizations, and the like. The compiled evidence 316 may include data such as, but not limited to, medical data, diagnostic data, hardware data, software data, patient data, patient population data, and/or other data. The compiled evidence 316 and/or central data structure 312 may include DMS', TPS, measurement stacks, and/or components thereof as described above, without limitation, with reference to FIGS. 1-2D.

The central data structure 312 may include one or more layers. In some instances, the central data structure 312 may include one or more layers as described above, without limitation, with reference to FIGS. 1-2D. Each component of digital measurement solutions and/or target solution profiles may be located in one or more layers of the central data structure 312. For instance and without limitation, the central data structure 312 may include a validation layer, an instrumentation layer, and/or a definition layer. A validation layer of the central data structure 312 may include data such as, but not limited to, regulatory intelligence and/or acceptance, clinical validation, and/or technical verification and/or analytical validation data. A validation layer of the central data structure 312 may include evidence to support a measurement stack is fit for purpose in a specific context of use. As a non-limiting example, a validation layer may include evidence that supports a measurement stack use in diagnosing Parkinson's disease. A validation layer of the central data structure 312 may be part of and/or incorporate one or more other layers of the central data structure 312, such as, without limitation, an instrumentation and/or definition layer.

An instrumentation layer of the central data structure 312 may include data such as, but not limited to, health data, algorithm data, raw data, measurement methods, and the like. Health data may include patient vital sign data, such as, but not limited to, heart rates, blood pressures, blood oxygen values, respiration rates, and/or other biological metrics of an individual. Algorithm data may include information on one or more algorithms and/or processes used to calculate, determine, and/or generate health data and/or measurement data of a patient. As a non-limiting example, algorithm data may include data of a specific EKG algorithm. Raw data may include information generated by one or more sensors. For instance, and without limitation, raw data may include raw sensor data of a heart rate monitor, respiratory monitor, blood oxygen monitor, and the like. Measurement methods may include one or more operations of obtaining health data and/or other data of an individual. Measurement methods may include one or more procedural steps used, hardware used, software used, clinical settings, patient population data, and the like, without limitation.

In some instances, an instrumentation layer of the central data structure may be part of and/or combined with a validation layer of the central data structure 312. An instrumentation layer of the central data structure 312 may include user-centric factors, hardware component details, software component details, and/or other components included in a capture and processing of data related to a concept being measured. User-centric factors may include, without limitation, ages, weights, heights, body compositions, ethnicities, blood types, and the like. Hardware component details may include, without limitation, processor information, sensor information, power supply information, and the like, of one or more sensors, medical devices, and/or other components having hardware. Software component details may include, without limitation, software versions, software types, software used, and the like, of one or more sensors and/or other measurement devices.

A definition layer of the central data structure 312 may include a concept of interest, meaning aspect of health, condition, and/or other parameters. A concept of interest may include areas of measurement of an individual, such as, without limitation, reflexes, attention, language, executive functioning, muscle strength, muscle atrophy, sleep patterns, digestive conditions, exercise, and/or other areas of measurement of an individual. A meaningful aspect of health (MAH) may include aspects of a condition in which a patient wants to improve and/or prevent. A condition may include any affliction a patient or individual may be experiencing, such as, without limitation, digestive issues, sleep apnea, motor imbalance, and the like.

In some instances, the central data structure 312 may include one or more measurement stacks. Measurement stacks may be as described above, without limitation in reference to FIG. 1. In some instances, each measurement stack may include one or more validation, instrumentation, and/or definition layers. In some instances, each measurement stack may include additional layers, such as meta data or other layers. A meta data layer may include a layer of data of one or more layers, such as a validation, instrumentation, and/or definition layer. In some instances, the central data structure 312 may act as a dossier. For instance, the central data structure 312 may be configured to store, update, and/or modify a plurality of measurement stacks, layers, DMS', TSP, and the like. The central data structure 312 may include data such as, but not limited to, input from one or more collaborators for a component and/or plurality of components of one or more standardized digital solutions, feedback from one or more parties, DMS data, TSP data, and the like.

Still referring to FIG. 3, in some instances, the processor 304 may be configured to receive a user request 320. The user request 320 may be received through user input, external computing devices, and/or other communication paths. For instance and without limitation, the user request 320 may be received through a mobile application, web browser, web portal, and the like. The user request 320 may be a request for a new standardized digital solution 324. The new standardized digital solution 324 may be a standardized digital solution for a diagnosis method, patient, disease, and the like, that has not been previously applied and/or recorded in the central data structure 312. The user request 320 may specify one or more disease, condition, patient, instrument, measurement, and/or other data. The processor 304 may query through the compiled evidence 316 of the central data structure 312 for data applicable to the user request 320. As a non-limiting example, the user request 320 may be for nocturnal scratch, and the processor 304 may query through the central data structure 312 for compiled evidence 316 of a nocturnal scratch.

Still referring to FIG. 3, in some instances, the processor 304 may be configured to receive additional data 328. The processor 304 may receive the additional data 328 through one or more external computing devices, user input at the apparatus 300, and the like, without limitation. The additional data 328 may include new data of one or more components of a DMS or TSP, such as, but not limited to, medical data, patient data, sensor data, disease data, and the like. The additional data 328 may be generated by an individual and/or organization performing one or more digital medical solutions. For instance, the additional data 328 may include sensor data of a heart rate sensor collected during execution of the new standardized digital solution 124. In some instances, the additional data 328 may include, without limitation, sensor data, hardware data, software data, patient data, diagnostic data, method data, disease data, and/or other types of data.

The processor 304 may be configured to update the central data structure 312 to incorporate the additional data 328. In some instances, updating the central data structure 312 to incorporate the additional data 328 may include modifying, replacing, and/or adding to the compiled evidence 316. For instance, and without limitation, the additional data 328 may include data of a previously approved DMS but for a new patient population, such as a pediatric patient population. The processor 304 may update the central data structure 312 and/or the compiled evidence 316 to include data of the new patient population. In some instances, the processor 304 may modify one or more DMS or TSP based on the additional data 328 to generate the new standardized digital solution 324. For instance, one or more components of a DMS or TSP may be modified and/or swapped with one or more other components of other DMS or TSP components. The new standardized digital solution 324 may be stored in the central data structure 312.

The processor 304 may be configured to leverage or otherwise use one or more components of the compiled evidence 316 to generate the new standardized digital solution 324. For instance, the processor 304 may determine compatible or otherwise useable data of the compiled evidence 316, such as, but not limited to, sensor data, hardware data, software data, patient data, disease data, and the like of a previously accepted standardized digital solution. For instance, and without limitation, the processor 304 may retrieve a measurement stack, DMS, and/or TSP having compatible data with requested data of the user request 320. The processor 304 may be configured to provide one or more measurement stacks from the central data structure 312 to a user in response to the user request 340.

In some instances, the processor 304 may determine an acceptability of the compiled evidence 316 based on an acceptability threshold or acceptability categorization. An acceptability threshold refers to a value or range of values that if met deems one or more aspects of a measurement stack as acceptable.

An acceptability threshold may include one or more values representing, but not limited to, a quantity of data, quality of data, recency of data, and the like. For instance, an acceptability threshold may include one or more standards that have been qualified and/or accepted by one or more regulators (in some instances, an analytical or technical standard). Standards may include performance levels, algorithm accuracies, sensor capabilities, and the like. An acceptability threshold may be specific to one or more components, sensors, algorithms, and the like. For instance, in a case of an acceptability threshold of a sensor, the acceptability threshold may include an algorithm requiring raw data measured at a specific frequency. In an instance, an acceptability threshold may include an interpretational need. For instance, an acceptability threshold may include an ability of a sensor to measure a sensitivity change at a specific level (e.g., quantitative level), for example a level that may help in clinical decision making. As a non-limiting example, an acceptability threshold may include a performance level of about 85% of an ideal value of an algorithm. As another non-limiting example, an acceptability threshold may include 100,000 measurements of a heart rate using a specific heart rate sensor. In this example, if a measurement stack utilizes 100,000 measurements of a heart rate using the specific heart rate sensor, then the specific heart rate sensor may be deemed acceptable for use in generation of the new standardized digital solution 324. In an instance, an acceptability threshold may be defined or determined at least in part by a qualification protocol for a DMS describing steps to be taken/requirements to qualify a particular solution.

The processor 304 may generate and/or retrieve one or more digital measurement templates, digital measurement solutions, and the like, based on the user request 320. In some instances, one or more digital measurement templates may be pre-populated with data that has been previously accepted by one or more individuals while having one or more blank fields for an individual to populate. The processor 304 may determine a reusability of a previously applied standardized digital solution for the user request 320. Reusability may refer to one or more properties of data and/or objects that may be used in multiple scenarios. As a non-limiting example, the processor 304 may determine a reusability of a DMS for sleep apnea of about 80% for a DMS for nocturnal scratch. For instance, in this example, a DMS for sleep apnea may have similar algorithms, sensors, methods, and/or instrumentation to that of a DMS for nocturnal scratch. In some instances, the processor 304 may generate the new standardized digital solution 324 based on a reused DMS of the central data structure 312 for a previously unapplied purpose. For instance, like the nocturnal scratch example above.

In other instances, the processor 304 may generate the new standardized digital solution 324 by adding to or modifying a previously applied DMS of the central data structure 312. The processor 304 may add the additional data 328 to a previously approved DMS of the central data structure 312 to generate an extended new standardized digital solution 324. An extended new standardized digital solution 324 may include a previously approved DMS with additional data, such as the additional data 328, incorporated. As a non-limiting example, a previously approved DMS for insomnia may be extended with additional data 328 including continuous air pressure pathway (CPAP) machine data, which may generate a new extended standardized digital solution 324 for sleep apnea.

Still referring to FIG. 3, in some instances, the processor 304 may identify one or more portions of the new standardized digital solution 324 that may have been altered and/or modified with the additional data 328. For instance, the processor 304 may display any data described throughout this disclosure through a graphical user interface (GUI). A GUI may include one or more icons, text elements, user input fields, and the like. A GUI may be displayed on a screen, such as, but not limited to, smartphones, tablets, monitors, and the like. In some instances, the processor 304 may highlight or otherwise mark the additional data 328 added to the new standardized digital solution 324. The processor 304 may generate and display one or more annotations of the additional data 328 added to the new standardized digital solution 324, such as, but not limited to, times, dates, hardware data, software data, medical professional data, feedback from one or more medical professionals, and the like. In some instances, the processor 304 may be configured to identify duplicate data, approved data, and the like. Identifying may include marking, highlighting, annotating, or otherwise marking data through a GUI display. For instance, and without limitation, the processor 304 may be configured to identify the new standardized digital solution 324 as being approved by one or more regulatory collaborators.

In some instances, prior to receiving the user request 320, the processor 304 may generate a digital environment that may include a plurality of components of standardized digital solutions. The digital environment may be accessible by a plurality of collaborators and/or users. In some instances, a digital environment may include a virtual network that may provide access to one or more digital files. In some instances, prior to receiving the user request 320, the processor 304 may generate a mission within a digital environment by establishing standardized roles for two or more collaborators. A mission may include a goal and/or objective for one or more persons. A standardized role may include a job of one or more collaborators. In some instances, the processor 304 may be configured to assign a primary role to a first of two or more collaborators and assign a secondary role to a second of two or more collaborators. A primary role may possess additional rights in comparison to a secondary role. In some instances, the processor 304 may assign an additional role to a primary and/or secondary role of a plurality of collaborators. An additional role may possess less rights than that of a primary and/or secondary role. In some instances, an additional role may be assigned to a funder, observer, technology provider, data partner, and the like, without limitation. Rights of one or more roles may include rights to access and/or view results developed from one or more missions. In some instances, rights of one or more roles may include rights to use results developed from one or more missions. In some instances, the processor 304 may be configured to select two or more collaborators for including in a mission prior to generating a mission. Generating a mission may include generating a private mission accessible only by two or more collaborators with define roles. In some instances, generating a mission includes generating a public mission accessible by one or more additional collaborators, such as, but not limited to, two or more collaborators. In some instances, prior to receiving input from one or more collaborators to define at least a component of the standardized new digital measurement solution 324, the processor 304 may be configured to receive an agreement from two or more collaborators on one or more criteria of a mission. Criteria of a mission may include terms and conditions of an agreement. In some instances, prior to receiving an agreement from each of two or more collaborators, the processor 304 may be configured to generate a pre-defined template identifying one or more criteria of a mission and provide the pre-defined template to each of the two or more collaborators. In some instances, generating a pre-defined template may include receiving an input from one or more collaborators for establishing a characteristic of a second role and incorporating the input from the one or more collaborators into a pre-defined template. In some instances,

In some instances, two or more collaborators may express a common interest, as one example advancing treatments of irritable bowel syndrome, without limitation. In some instances, the processor 304 may be configured to receive input from one or more collaborators towards a component of the new standardized digital solution 324. The processor 304 may be configured to provide access to at least a portion of the central data structure 312 to one or more collaborators. In some instances, the processor 304 may be configured to receive feedback from a regulator collaborator specific for a received input contributing towards at least a component of the new standardized digital solution 324. Feedback from a regulatory collaborator may include a regulatory acceptance of at least a component of the new standardized digital solution 324. In some instances, feedback from a regulatory collaborator may include one or more questions or requests pertaining to at least a component of the new standardized digital solution 324. In some instances, the processor 304 may be configured to receive additional input from two or more collaborators responsive to feedback from a regulatory collaborate. The processor 304 may document additional input form two or more collaborators in the central data structure 312 accessible within a mission and/or goal. In some instances, the processor 304 may be configured to publish the central data structure 312 outside of a first mission for use in one or more subsequent mission.

In some instances, the processor 304 may be configured to display a version history of one or more DMSs. A version history may include a last used date, last updated date, and/or other data of a DMS. The processor 304 may be configured to generate a collection of one or more measurement stacks of one or more DMS'. A collection of one or more measurement stacks may be generated based on a condition, disease, patient population, and the like. For instance, and without limitation, the processor 304 may generate a collection of measurement stacks for teenage asthma. A collection of measurement stacks may be generated for one or more contexts of use (COU).

### Acceptability of Lavers of a Measurement Stack

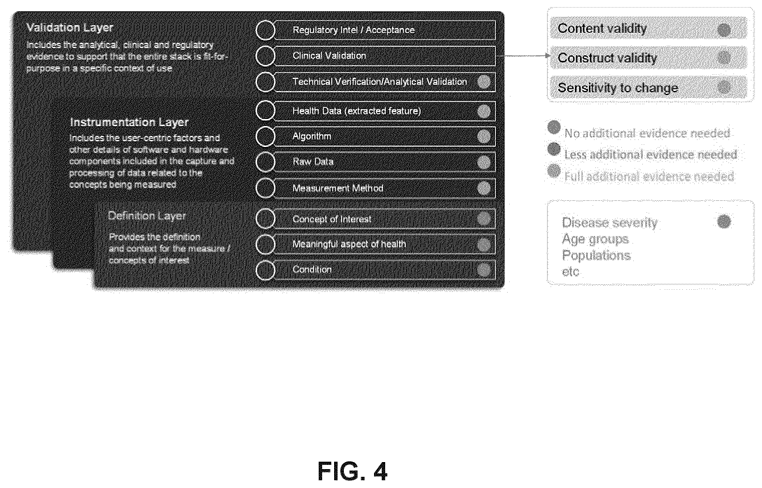

FIG. 4 is an exemplary instance of a graphical user interface (GUI) showing various levels of acceptability of layers of a measurement stack. A measurement stack includes one or more layers, such as, but not limited to, a validation, instrumentation, and/or definition layer, such as described above with reference to FIGS. 1-3. A processor and/or computing device, such as the processor 304 described above with reference to FIG. 3, is configured to calculate or otherwise determine an acceptability of one or more components of each layer of a measurement stack. An acceptability may refer to a reusability and one or more components of one or more layers for various purposes. For instance, one or more components of one or more layers of a measurement stack may be reused for a different disease, condition, patient, sensing device, and the like. For instance, a validation layer may have components of, without limitation, regulatory intelligence and/or acceptance, clinical validation, and/or technical verification/analytical validation. Each of the above listed components may have a corresponding acceptability score which may be determined based on an acceptability criteria (or acceptability threshold). Acceptability criteria may include, without limitation, quality of data, quantity of data, recency of data, and/or other criteria. For instance, each component of a layer may have data respective of that component. As a non-limiting example, in an instrumentation layer, components of health data, algorithm data, raw data, and/or measurement method data may all have corresponding data.

In some instances, an acceptability criteria may include a threshold value, such as, but not limited to, 70%, 80%, 90% and the like. In some instances, an acceptability criteria may include two or more acceptability criterion. For instance, for a definition layer, acceptability criteria may include both a recency of the data and a quantity of the data respective to a condition component of the definition layer. In some instances, acceptability criteria may be specific to one or more components of one or more layers of a measurement stack. For instance, in a validation layer, acceptability criteria may include a recency of the data of one or more components of the validation layer, while in an instrumentation layer acceptability criteria may include a quantity of data of one or more components of the instrumentation layer. In some instances, acceptability criteria may be pre-defined by one or more user inputs. In other instances, a computing device and/or processor, such as processor 304 described above, may be configured to calculate an acceptability criteria based on one or more factors. For instance, and without limitation, a computing device and/or processor may be configured to determine an acceptability criteria to include a quantity of data for a wrist-worn heart rate sensor of a raw data component of an instrumentation layer based on data of one or more databases, such as central data structure 312 as described above with reference to FIG. 3.

In some instances, one or more components of one or more layers of a measurement stack may partially or fully meet an acceptability criteria. In some instances, one or more components of one or more layers of a measurement stack may not meet an acceptability criteria. Each component of a layer of a measurement stack may meet an acceptability criteria fully, partially, and/or a combination thereof. For instance and without limitation, a first component of a definition layer may meet an acceptability criteria fully while a second component of the definition layer may only partially meet an acceptability criteria. A processor and/or computing device may categorize acceptability of one or more components of one or more layers.

Categorization may include sorting and/or placing one or more components into categories, such as, but not limited to, valid data, verified data, construct validity (e.g., how well a solution measures an intended concept), sensitivity to change, and the like. With respect to construct validity, for example and without limitation, when measuring a number of steps of a patient, acceleration and/or other measurements may be taken to determine a number of steps. Continuing this example, a construct validity of the measurement of a number of steps may refer to how well the acceleration and/or other measurements reflect the accuracy of the number of steps taken. In some instances, categorization may include content validity, e.g., whether a measurement is suitable for a given concept. With respect to content validity, for instance and without limitation, a concept may be physical activity and a measurement may be a number of steps and a content validity may be how well the measurement of a number of steps reflects physical activity of a patient, as other measurement such as gait speed may be used as may be appropriate for a particular purpose.

Sensitivity to change refers to, e.g., how a measurement may reflect underlying changes in a concept. For example, asking a patient about their level of physical activity through a questionnaire may have poor sensitivity to change since the changes would have to be quite substantial for the patient to rate the changes in the questionnaire, whereas a direct measure can be more sensitive. In some instances, each component of each layer of a measurement stack may be categorized to evidence-based categories of “no additional evidence needed”, “less additional evidence needed”, and/or “full additional evidence needed”. A “no additional evidence needed” category may include a grouping of one or more sets of data that are ready for reuse. For instance, one or more sets of data may meet one or more acceptability criteria, such as a quantity, recency, and/or quality of data. A “no additional evidence needed” category may be set through user input and/or determined by one or more computing devices. In some instances, a “less additional evidence needed” category may include a group of one or more sets of data that partially meet one or more acceptability criteria. For instance, one or more sets of data of a component may partially meet one or more acceptability criteria, such as, without limitation, a recency, quantity, and/or quality of data. In some instances, a “full additional evidence needed” category may include a group of one or more sets of data of a component that fails to meet one or more acceptability criteria, such as, but not limited to, a recency, quantity, and/or quality of data.

In some instances, acceptability of one or more components may be color coded. For instance, a measurement stack and each component thereof may be displayed through a GUI. A colored circle may be positioned on a left, right, top, and/or bottom of a component of a measurement stack displayed through a GUI. A color of a colored circle may represent a category of data of a component. For instance, a green color of a circle may correspond to a “no additional evidence needed” category, a blue color may correspond to a “less additional evidence needed” category, and an orange color may correspond to a “full additional evidence needed” category, without limitation, A color code may include any color, such as red, blue, green, yellow, and/or a combination thereof. In some instances, a color code may be used for each layer of a measurement stack. For instance, a validation layer may be displayed with a green circle, an instrumentation layer may be displayed with a blue circle, and a definition layer may be displayed with an orange circle, without limitation. In other instances, each component of each layer of a measurement stack may be displayed with a corresponding colored circle. In some instances, one or more annotations of one or more components may be displayed. Annotations may include, without limitation, text, icons, colors, and the like. In some instances, annotations may include additional data regarding an acceptability of one or more components of a layer of a measurement stack. For instance, and without limitation, a clinical validation component of a validation layer may be displayed with an annotation, such as a text box, that may show various parts of the clinical validation component that meet one or more acceptability criteria. Continuing this non-limiting example, the annotations of the clinical validation component may include a “content validity” text box with a green circle corresponding to a “no additional evidence needed” category, a “construct validity” box with an orange circle corresponding to a “full additional evidence needed” category, and/or a “sensitivity to change” text box with an orange circle corresponding to the “full additional evidence needed” category. A user may click on one or more components of one or more layers of a measurement stack, which may cause a GUI to display one or more annotation boxes of one or more components. Of course, the foregoing is merely exemplary, and different instances may use different colors, or other pattens, fonts, or other distinguishing visual features, layouts, or so on for different criteria or indications of acceptability.

In some instances, one or more layers may still be reused and/or extended despite having one or more components of the one or more layers failing to meet an acceptability criteria. As a non-limiting example, a concept of interest component and a condition component of a definition layer may meet an acceptability criteria, partially or fully, while a meaningful aspect of health component of the definition layer may fail to meet an acceptability criteria. Continuing this example, the definition layer may still be acceptable for reuse for various purposes. In some instances, a computing device and/or processor may determine a total acceptability of one or more layers. A total acceptability may include a sum of two or more components of a layer. For instance, in the above example of the definition layer, the definition layer may have a total acceptability of 70%.

### Flowchart for Reusability and Extensions

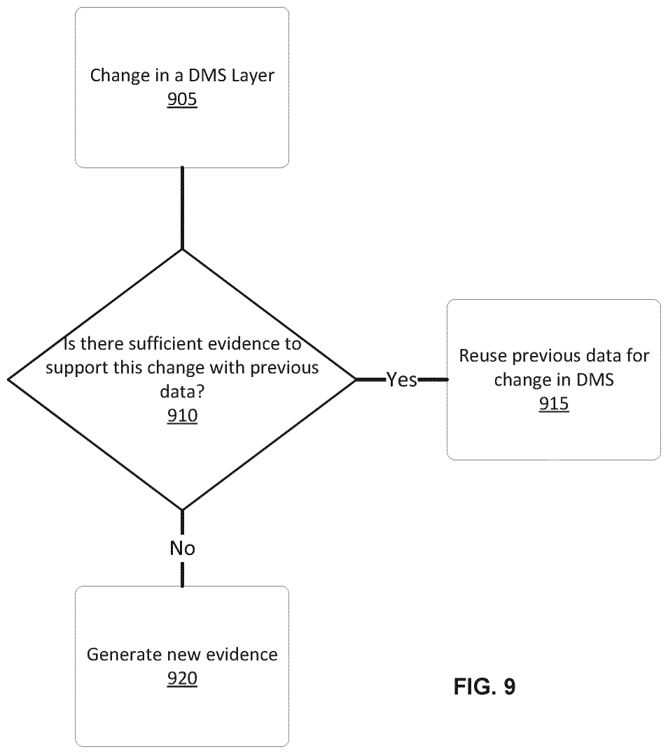

Referring now to FIG. 9, an exemplary instance of a flowchart is presented. Flowchart 900 may be implemented in any process, system, and/or method as described throughout this disclosure. A computing device may be configured to perform one or more steps of the flowchart 900. In some instances, the flowchart 900 may provide for a comparison of one or more aspects of a DMS against one or more criterion. Criteria may include, without limitation, age of data, quantity of data, quality of data, algorithm likeness, measurement method likeness, condition likeness, and the like, without limitation. Criteria may be received by user input and/or through one or more external computing devices. Aspects may include, without limitation, regulatory intelligence and/or acceptance, clinical validation, technical verification/analytical validation, health data, algorithms, raw data, measurement methods, concepts of interest, meaning aspects of health, conditions, and the like of a DMS. The flowchart 900 may allow for a determination of a reusability of one or more aspects of a DMS, an extension of one or more aspects of a DMS, and the like. A reusability may include one or more parts of one or more layers of a DMS that may be used for a different population, condition, measurement method, algorithm, and the like. An extension of one or more aspects of a DMS may include additional data that may be needed for a different patient population, condition, measurement method, and the like.

In some instances, the flowchart 900 may be performed automatically through one or more computing devices. For instance, a computing device may be configured to compare one or more aspects of a DMS and/or TSP to one or more criteria of the flowchart 900 and generate an output of a reusability, extension, or other function of an aspect of a DMS and/or TSP. In some instances, a computing device may utilize a machine learning model to generate a flowchart 900 and/or other comparison procedure. A machine learning model may decide which path to take in the flowchart 900. A machine learning model may be trained with training data correlating one or more aspects of a DMS and one or more criteria to reusability, extensions, and/or other functions of the one or more aspects of the DMS. Training data may be received through user input, external computing devices, and/or previous iterations of processing. A machine learning model may be configured to input data of one or more validation, instrumentation, and/or definition layers and output one or more determinations of a reusability, extension, and/or other function of one or more layers. An artificial intelligence, machine learning model, or other machine process may be implemented by a computing device. In some instances, an artificial intelligence and/or machine learning model may be configured to identify one or more components that may be acceptable for repurposing for different interests. In an instance, an artificial intelligence and/or machine learning model may be configured to identify one or more matches between an ongoing mission with a new interest/mission of a DMS. For instance, a clustering algorithm may be used to identify potential matches between an ongoing mission with a new interest and/or mission. A computing device may be configured to update criteria for one or more validation, instrumentation, and/or definition layers of one or more DMS. In some instances, a computing device may be configured to update criteria by utilizing a machine learning model. Criteria of the flowchart 900 or other determination process may be specific to one or more aspects of a DMS, such as in a definition layer of the DMS. Aspects of a definition layer may include an aspect of health, condition, population, and the like.

At step 905, a change in a component of a standardized digital solution (e.g., a DMS layer) is made. A change may include any aspect of any layer of a standardized digital solution (e.g., a TSP or a DMS), such as, but not limited to, regulatory intelligence and/or acceptance, clinical validation, technical verification/analytical validation, health data, algorithms, raw data, measurement methods, concepts of interest, meaning aspects of health, conditions, and the like of a TSP or DMS. For instance, a change in a TSP or DMS layer may include a change in a disease or condition to be treated.

At step 910, a comparison of the change in the TSP or DMS layer to previously used data is made. Previously used data may include evidence of one or more aspects of one or more DMS layers. In some instances, a computing device may utilize a loss function to determine a threshold difference between the change in the DMS layer and previously used evidence/data. In some instances, a threshold difference may include, without limitation, about 1% to about 100%. For instance, a threshold difference of about 15% may be used to determine if previously used data is sufficient to cover the change in the DMS layer.

As an example, assume a previously approved DMS. In various instances, a change to the previously approved DMS may be a change to the meaningful aspect of health component (e.g., a new meaningful aspect of health is introduced). As such, the additional components of the DMS (e.g., the concept of interest, the measurement method, the raw data, the algorithm, the health data, the technical verification/analytical validation, the clinical validation, and/or the regulatory intelligence/acceptance) undergo an evaluation to determine which of the components can be re-used and which components are to undergo a full re-validation or a partial re-validation. As an example, referring to the measurement method component, the introduction of a new meaningful aspect of health triggers an evaluation of the measurement method component to determine whether the measurement method already captures the relevant data for the new meaningful aspect of health. If the measurement method successfully captures the relevant data for the new meaningful aspect of health, then the measurement method component can be re-used without undergoing further evaluation (e.g., step 915). In one scenario, if the measurement method successfully captures the relevant data for the new meaningful aspect of health, then the measurement method undergoes only a partial validation, without needing to undergo a full re-validation. If the measurement method fails to capture the relevant data for the new meaningful aspect of health, then the measurement method component undergoes a full re-validation by generating new evidence (e.g., step 920).

As another example, referring to the technical verification/analytical validation component, the introduction of a new meaningful aspect of health triggers an evaluation of the technical verification/analytical validation component. As described herein, in various instances, an analytical validation component may perform an analytical validation of device specifications, algorithms, and health data output and ensures that the data meets requisite sensitivity, specificity, and/or reliability requirements. Thus, an evaluation of the technical verification/analytical validation component can include determining whether the device specifications, algorithms, and health data output remain within the required sensitivity, specificity, and/or reliability requirements. In some instances, if it is determined that the required sensitivity, specificity, and/or reliability requirements are not met, the technical verification/analytical validation component will undergo a full re-validation (e.g., step 920). In some instances, if it is determined that the required sensitivity, specificity, and/or reliability requirements are met, the technical verification/analytical validation component can undergo a partial re-validation or can automatically be reused (e.g., step 915).

At step 915, if there is sufficient evidence to support the change in the DMS layer with previous data, the previous data may be reused for the change in the DMS layer. Reused data may include, without limitation, algorithms, sensors, methods of measurement, concepts of interest, conditions, analytical validation, and the like.

At step 920, if there is not sufficient evidence to support the change in the DMS layer, new evidence may be generated. New evidence generation may produce an extension of one or more layers, stacks, and the like, of a DMS. New evidence generation may allow future changes to one or more DMS layers to reuse the data generated as part of the extension of the DMS layer.

### Diseases and Conditions

Disclosed herein are TSPs and DMSs that are built and implemented for specific diseases or conditions. In various instances, the disease can be, for example, a cancer, inflammatory disease, neurodegenerative disease, neurological disease, autoimmune disorder, neuromuscular disease, metabolic disorder (e.g., diabetes), cardiac disease, or fibrotic disease.

In various instances, the cancer can be any one of lung bronchioloalveolar carcinoma (BAC), bladder cancer, a female genital tract malignancy (e.g., uterine serous carcinoma, endometrial carcinoma, vulvar squamous cell carcinoma, and uterine sarcoma), an ovarian surface epithelial carcinoma (e.g., clear cell carcinoma of the ovary, epithelial ovarian cancer, fallopian tube cancer, and primary peritoneal cancer), breast carcinoma, non-small cell lung cancer (NSCLC), a male genital tract malignancy (e.g., testicular cancer), retroperitoneal or peritoneal carcinoma, gastroesophageal adenocarcinoma, esophagogastric junction carcinoma, liver hepatocellular carcinoma, esophageal and esophagogastric junction carcinoma, cervical cancer, cholangiocarcinoma, pancreatic adenocarcinoma, extrahepatic bile duct adenocarcinoma, a small intestinal malignancy, gastric adenocarcinoma, cancer of unknown primary (CUP), colorectal adenocarcinoma, esophageal carcinoma, prostatic adenocarcinoma, kidney cancer, head and neck squamous carcinoma, thymic carcinoma, non-melanoma skin cancer, thyroid carcinoma (e.g., papillary carcinoma), a head and neck cancer, anal carcinoma, non-epithelial ovarian cancer (non-EOC), uveal melanoma, malignant pleural mesothelioma, small cell lung cancer (SCLC), a central nervous system cancer, a neuroendocrine tumor, and a soft tissue tumor. For example, in certain instances, the cancer is breast cancer, non-small cell lung cancer, bladder cancer, kidney cancer, colon cancer, and melanoma.

In various instances, the inflammatory disease can be any one of acute respiratory distress syndrome (ARDS), acute lung injury (ALI), alcoholic liver disease, allergic inflammation of the skin, lungs, and gastrointestinal tract, allergic rhinitis, ankylosing spondylitis, asthma (allergic and non-allergic), atopic dermatitis (also known as atopic eczema), atherosclerosis, celiac disease, chronic obstructive pulmonary disease (COPD), pulmonary arterial hypertension (PAH), chronic respiratory distress syndrome (CRDS), colitis, dermatitis, diabetes, eczema, endocarditis, fatty liver disease, fibrosis (e.g., idiopathic pulmonary fibrosis, scleroderma, kidney fibrosis, and scarring), food allergies (e.g., allergies to peanuts, eggs, dairy, shellfish, tree nuts, etc.), gastritis, gout, hepatic steatosis, hepatitis, inflammation of body organs including joint inflammation including joints in the knees, limbs or hands, inflammatory bowel disease (IBD) (including Crohn's disease or ulcerative colitis), intestinal hyperplasia, irritable bowel syndrome, juvenile rheumatoid arthritis, liver disease, metabolic syndrome, multiple sclerosis, myasthenia gravis, neurogenic lung edema, nephritis (e.g., glomerular nephritis), non-alcoholic fatty liver disease (NAFLD) (including non-alcoholic steatosis and non-alcoholic steatohepatitis (NASH)), obesity, prostatitis, psoriasis, psoriatic arthritis, rheumatoid arthritis (RA), sarcoidosis sinusitis, splenitis, seasonal allergies, sepsis, systemic lupus erythematosus, uveitis, and UV-induced skin inflammation.

In various instances, the neurodegenerative disease can be any one of Alzheimer's disease, Parkinson's disease, traumatic CNS injury, Down Syndrome (DS), glaucoma, amyotrophic lateral sclerosis (ALS), frontotemporal dementia (FTD), and Huntington's disease. In addition, the neurodegenerative disease can also include Absence of the Septum Pellucidum, Acid Lipase Disease, Acid Maltase Deficiency, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS, Alexander Disease, Alper's Disease, Alternating Hemiplegia, Anencephaly, Aneurysm, Angelman Syndrome, Angiomatosis, Anoxia, Antiphosphipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Autism, Autonomic Dysfunction, Barth Syndrome, Batten Disease, Becker's Myotonia, Behcet's Disease, Bell's Palsy, Benign Essential Blepharospasm, Benign Focal Amyotrophy, Benign Intracranial Hypertension, Bemhardt-Roth Syndrome, Binswanger's Disease, Blepharospasm, Bloch-Sulzberger Syndrome, Brachial Plexus Injuries, Bradbury-Eggleston Syndrome, Brain or Spinal Tumors, Brain Aneurysm, Brain injury, Brown-Sequard Syndrome, Bulbospinal Muscular Atrophy, Cadasil, Canavan Disease, Causalgia, Cavemomas, Cavernous Angioma, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Chorea, Chronic Inflammatory Demyelinating Polyneuropathy (CIDP), Coffin Lowry Syndrome, Colpocephaly, Congenital Facial Diplegia, Congenital Myasthenia, Congenital Myopathy, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Dancing Eyes-Dancing Feet Syndrome, Dandy-Walker Syndrome, Dawson Disease, Dementia, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyssynergia Cerebellaris Myoclonica, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry Disease, Fahr's Syndrome, Fainting, Familial Dysautonomia, Familial Hemangioma, Familial Periodic Paralyzes, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Hemicrania Continua, Hemifacial Spasm, Hemiplegia Alterans, Hereditary Neuropathy, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydromyelia, Hypernychthemeral Syndrome, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaac's Syndrome, Joubert syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus, Lyme Disease, Machado-Joseph Disease, Macrencephaly, Melkersson-Rosenthal Syndrome, Meningitis, Menkes Disease, Meralgia Paresthetica, Metachromatic Leukodystrophy, Microcephaly, Migraine, Miller Fisher Syndrome, Mini-Strokes, Mitochondrial Myopathies, Motor Neuron Diseases, Moyamoya Disease, Mucolipidoses, Mucopolysaccharidoses, Multiple sclerosis (MS), Multiple System Atrophy, Muscular Dystrophy, Myasthenia Gravis, Myoclonus, Myopathy, Myotonia, Narcolepsy, Neuroacanthocytosis, Neurodegeneration with Brain Iron Accumulation, Neurofibromatosis, Neuroleptic Malignant Syndrome, Neurosarcoidosis, Neurotoxicity, Nevus Cavernosus, Niemann-Pick Disease, Non 24 Sleep Wake Disorder, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Perineural Cysts, Periodic Paralyzes, Peripheral Neuropathy, Periventricular Leukomalacia, Pervasive Developmental Disorders, Pinched Nerve, Piriformis Syndrome, Plexopathy, Polymyositis, Pompe Disease, Porencephaly, Postherpetic Neuralgia, Postinfectious Encephalomyelitis, Post-Polio Syndrome, Postural Hypotension, Postural Orthostatic Tachyardia Syndrome (POTS), Primary Lateral Sclerosis, Prion Diseases, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Saint Vitus Dance, Sandhoff Disease, Schizencephaly, Septo-Optic Dysplasia, Shingles, Shy-Drager Syndrome, Sjogren's Syndrome, Sleep Apnea, Sleeping Sickness, Sotos Syndrome, Spasticity, Spinal Cord Infarction, Spinal Cord Injury, Spinal Cord Tumors, Spinocerebellar Atrophy, Spinocerebellar Degeneration, Stiff-Person Syndrome, Striatonigral Degeneration, Stroke, Sturge-Weber Syndrome, SUNCT Headache, Syncope, Syphilitic Spinal Sclerosis, Syringomyelia, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tinnitus, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wemicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

In various instances, the autoimmune disease or disorder can be any one of arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis deformans, polyarthritis chronica primaria, reactive arthritis, and ankylosing spondylitis; inflammatory hyperproliferative skin diseases; psoriasis, such as plaque psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia pemiciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcomeal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic fasciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; choroiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodo sum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired splenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic reperfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis. In particular instances, the autoimmune disorder in the subject can include one or more of: systemic lupus erythematosus (SLE), lupus nephritis, chronic graft versus host disease (cGVHD), rheumatoid arthritis (RA), Sjogren's syndrome, vitiligo, inflammatory bowed disease, and Crohn's Disease. In particular instances, the autoimmune disorder is systemic lupus erythematosus (SLE). In particular instances, the autoimmune disorder is rheumatoid arthritis.

Exemplary metabolic disorders include, for example, diabetes, insulin resistance, lysosomal storage disorders (e.g., Gauchers disease, Krabbe disease, Niemann Pick disease types A and B, multiple sclerosis, Fabry's disease, Tay Sachs disease, and Sandhoff Variant A, B), obesity, cardiovascular disease, and dyslipidemia. Other exemplary metabolic disorders include, for example, 17-alpha-hydroxylase deficiency, 17-beta hydroxysteroid dehydrogenase 3 deficiency, 18 hydroxylase deficiency, 2-hydroxyglutaric aciduria, 2-methylbutyryl-CoA dehydrogenase deficiency, 3-alpha hydroxyacyl-CoA dehydrogenase deficiency, 3-hydroxyisobutyric aciduria, 3-methylcrotonyl-CoA carboxylase deficiency, 3-methylglutaconyl-CoA hydratase deficiency (AUH defect), 5-oxoprolinase deficiency, 6-pyruvoyl-tetrahydropterin synthase deficiency, abdominal obesity metabolic syndrome, abetalipoproteinemia, acatalasemia, aceruloplasminemia, acetyl CoA acetyltransferase 2 deficiency, acetyl-carnitine deficiency, acrodermatitis enteropathica, adenine phosphoribosyltransferase deficiency, adenosine deaminase deficiency, adenosine monophosphate deaminase 1 deficiency, adenylosuccinase deficiency, adrenomyeloneuropathy, adult polyglucosan body disease, albinism deafness syndrome, alkaptonuria, Alpers syndrome, alpha-1 antitrypsin deficiency, alpha-ketoglutarate dehydrogenase deficiency, alpha-mannosidosis, aminoacylase 1 deficiency, anemia sideroblastic and spinocerebellar ataxia, arginase deficiency, argininosuccinic aciduria, aromatic L-amino acid decarboxylase deficiency, arthrogryposis renal dysfunction cholestasis syndrome, Arts syndrome, aspartylglycosaminuria, atypical Gaucher disease due to saposin C deficiency, autoimmune polyglandular syndrome type 2, autosomal dominant optic atrophy and cataract, autosomal erythropoietic protoporphyria, autosomal recessive spastic ataxia 4, Barth syndrome, Bartter syndrome, Bartter syndrome antenatal type 1, Bartter syndrome antenatal type 2, Bartter syndrome type 3, Bartter syndrome type 4, Beta ketothiolase deficiency, biotinidase deficiency, Bjornstad syndrome, carbamoyl phosphate synthetase 1 deficiency, carnitine palmitoyl transferase 1A deficiency, carnitine-acylcarnitine translocase deficiency, carnosinemia, central diabetes insipidus, cerebral folate deficiency, cerebrotendinous xanthomatosis, ceroid lipofuscinosis neuronal 1, Chanarin-Dorfman syndrome, Chediak-Higashi syndrome, childhood hypophosphatasia, cholesteryl ester storage disease, chondrocalcinosisc, chylomicron retention disease, citrulline transport defect, congenital bile acid synthesis defect, type 2, Crigler Najjar syndrome, cytochrome c oxidase deficiency, D-2-hydroxyglutaric aciduria, D-bifunctional protein deficiency, D-glycericacidemia, Danon disease, dicarboxylic aminoaciduria, dihydropteridine reductase deficiency, dihydropyrimidinase deficiency, diabetes insipidus, dopamine beta hydroxylase deficiency, Dowling-Degos disease, erythropoietic uroporphyria associated with myeloid malignancy, Familial chylomicronemia syndrome, Familial HDL deficiency, Familial hypocalciuric hypercalcemia type 1, Familial hypocalciuric hypercalcemia type 2, Familial hypocalciuric hypercalcemia type 3, Familial LCAT deficiency, Familial partial lipodystrophy type 2, Fanconi Bickel syndrome, Farber disease, fructose-1,6-bisphosphatase deficiency, gamma-cystathionase deficiency, Gaucher disease, Gilbert syndrome, Gitelman syndrome, glucose transporter type 1 deficiency syndrome, glutamine deficiency, congenital, Glutaric acidemia. glutathione synthetase deficiency, glycine N-methyltransferase deficiency, Glycogen storage disease hepatic lipase deficiency, homocysteinemia, Hurler syndrome, hyperglycerolemia, Imerslund-Grasbeck syndrome, iminoglycinuria, infantile neuroaxonal dystrophy, Keams-Sayre syndrome, Krabbe disease, lactate dehydrogenase deficiency, Lesch Nyhan syndrome, Menkes disease, methionine adenosyltransferase deficiency, mitochondrial complex deficiency, muscular phosphorylase kinase deficiency, neuronal ceroid lipofuscinosis, Niemann-Pick disease type A, Niemann-Pick disease type B, Niemann-Pick disease type C1, Niemann-Pick disease type C2, ornithine transcarbamylase deficiency, Pearson syndrome, Perrault syndrome, phosphoribosylpyrophosphate synthetase superactivity, primary camitine deficiency, hyperoxaluria, purine nucleoside phosphorylase deficiency, pyruvate carboxylase deficiency, pyruvate dehydrogenase complex deficiency, pyruvate dehydrogenase phosphatase deficiency, yruvate kinase deficiency, Refsum disease, diabetes mellitus, Scheie syndrome, Sengers syndrome, Sialidosis Sjogren-Larsson syndrome, Tay-Sachs disease, transcobalamin 1 deficiency, trehalase deficiency, Walker-Warburg syndrome, Wilson disease, Wolfram syndrome, and Wolman disease.

### Non-transitory Computer Readable Medium

Also provided herein is a computer readable medium comprising computer executable instructions configured to implement any of the methods described herein. In various instances, the computer readable medium is a non-transitory computer readable medium. In some instances, the computer readable medium is a part of a computer system (e.g., a memory of a computer system). The computer readable medium can comprise computer executable instructions for performing methods disclosed herein, such as methods for building, maintaining, implementing, and providing standardized solutions (e.g., DMSs and TSPs).

### Computing Device

The methods described above, including the methods of building, maintaining, implementing, and providing standardized solutions (e.g., DMSs and TSPs) are, in some instances, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

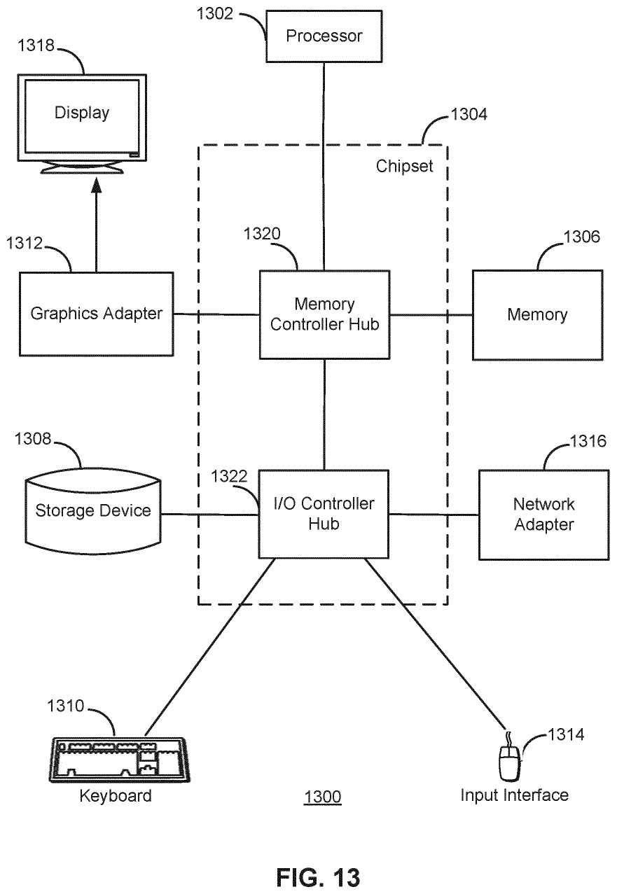

FIG. 13 illustrates an example computing device 900 for implementing system and methods described throughout this disclosure. In some instances, the computing device 1300 includes at least one processor 1302 coupled to a chipset 1304. The chipset 1304 includes a memory controller hub 1320 and an input/output (I/O) controller hub 1322. A memory 1306 and a graphics adapter 1312 are coupled to the memory controller hub 1320, and a display 1318 is coupled to the graphics adapter 1312. A storage device 1308, an input interface 1314, and network adapter 1316 are coupled to the I/O controller hub 1322. Other instances of the computing device 1300 have different architectures.

The storage device 1308 is a non-transitory computer-readable storage medium such as a hard drive, compact disk read-only memory (CD-ROM), DVD, or a solid-state memory device. The memory 1306 holds instructions and data used by the processor 1302. The input interface 1314 is a touch-screen interface, a mouse, track ball, or other type of input interface, a keyboard, or some combination thereof, and is used to input data into the computing device 1300. In some instances, the computing device 1300 may be configured to receive input (e.g., commands) from the input interface 1314 via gestures from the user. The graphics adapter 1312 displays images and other information on the display 1318. As an example, the display 1318 can show a catalog of standardized solutions (e.g., DMSs and/or TSPs). The network adapter 1316 couples the computing device 1300 to one or more computer networks.

The computing device 1300 is adapted to execute computer program modules for providing functionality described herein. As used herein, the term “module” refers to computer program logic used to provide the specified functionality. Thus, a module can be implemented in hardware, firmware, and/or software. In one instance, program modules are stored on the storage device 1308, loaded into the memory 1306, and executed by the processor 1302.

The types of computing devices 1300 can vary from the instances described herein. For example, the computing device 1300 can lack some of the components described above, such as graphics adapters 1312, input interface 1314, and displays 1318. In some instances, a computing device 1300 can include a processor 1302 for executing instructions stored on a memory 1306.

### EXAMPLES

Below are examples of specific instances for carrying out the present invention. The examples are offered for illustrative purposes only and are not intended to limit the scope of the present invention in any way. Efforts have been made to ensure accuracy with respect to numbers used, but some experimental error and deviation should be allowed for.

### Example 1: Acceptability of Layers of a Measurement Stack

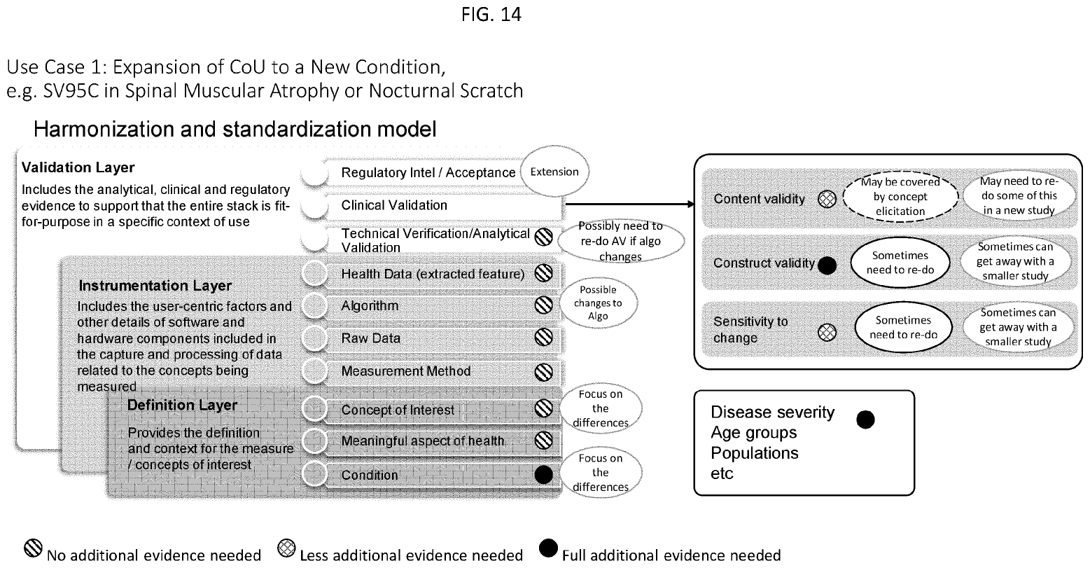

FIG. 14 depicts a first example of an acceptability level of a digital measurement solution. Specifically, the first example is a use case of an expansion of a context of use to a new condition in Spinal Muscular Atrophy or Nocturnal Scratch. This case uses the SV95C clinical assessment, which is a wearable-derived digital clinical outcome assessment qualified by the European Medicines agency in Duchenne Muscular Dystrophy (DMD), in Nocturnal Scratch. In other words, here, the digital measurement solution is applying the SV95C clinical assessment to a new condition/disease. The digital measurement solution has a validation layer, instrumentation layer, and a definition layer. The validation layer includes a regulatory intelligence/acceptance aspect, a clinical validation aspect, and a technical verification/analytical validation aspect. Each of these aspects are categorized as no additional evidence needed. The clinical validation aspect has three sub-layers, a content validity, construct validity, and sensitivity to change layer. These three sub-layers may be applied to other layers of the DMS. The content validity layer is categorized as having less additional evidence needed. This categorization comes from data in the content validity being covered by concept elicitation but also needed to redo some content validity in a new study. The construct validity layer is categorized as having full additional evidence need. This categorization comes from the data of the clinical validation layer that is flagged as needed to be re-done, such as with a smaller study group. The sensitivity to change layer is categorized as need partial additional evidence. This categorization comes from the data of the clinical validation having one or more portions that sometimes require re-doing, such as with a small study group.

The technical verification/analytical validation layer is marked as not needing additional evidence. For instance, it can be possible to use a same algorithm in the analytical validation layer. Sometimes, if an algorithm changes, the analytical validation layer can be re-calculated. The instrumentation layer has a health data, algorithm, raw data, and measurement method layer. Each of these layers are categorized as not needing additional evidence. However, there can be possible changes to the algorithm of the algorithm layer. The definition layer has a concept of interest, meaningful aspect of health, and condition layer. The concept of interest and meaningful aspect of health layers are categorized as not needing additional evidence. The condition layer is categorized as needing full additional evidence. This can be attributed to the expansion of the DMS from a condition of Muscular Atrophy to Nocturnal Scratch. Full additional evidence is needed to expand the DMS for use in Nocturnal Scratch while the other layers of the DMS can still be applied to Nocturnal Scratch.

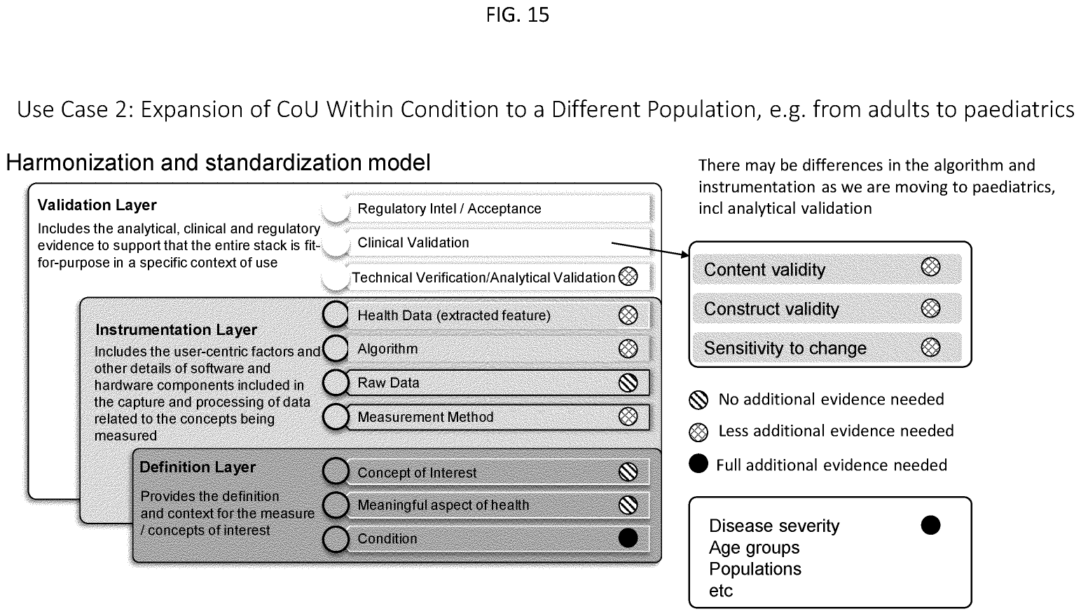

FIG. 15 depicts a second example of acceptability levels of a digital measurement solution. Specifically, the second example is an expansion of a context of use within a condition to a different population, from adults to pediatrics. Here, there may be differences in instrumentation and algorithms in analytical validation from an adult population to pediatrics. As described above, the DMS has a validation layer, instrumentation layer, and definition layer. The validation layer has a regulatory intelligence layer, clinical validation layer, and technical verification/analytical validation layer. In this example, each of the three layers are categorized as needing less additional evidence. For instance, a content validity, construct validity, and sensitivity to change of each layer are all marked as needing less additional evidence. The instrumentation layer has a health data, algorithm, raw data, and measurement method layer. The health data, algorithm, and measurement method layers of the instrumentation layer are categorized as needing less additional evidence. The raw data of the instrumentation layer is categorized as not needing additional evidence. The raw data layer may not need additional evidence where performance requirements and/or evidence for one or more sensors already are sufficient, such as accelerometers, gyroscopes, and the like. However, the algorithm, health data, and measurement method need some additional evidence to account for a different population of patient. The definition layer has a concept of interest, meaning aspect of health, and condition layer. The concept of interest layer and the meaningful aspect of health layers are categorized as not needing additional evidence, while the condition layer is categorized as needing full additional evidence. The condition layer is categorized as needing full additional evidence due to the new population, such as to account for disease severity, age groups, populations, and the like.

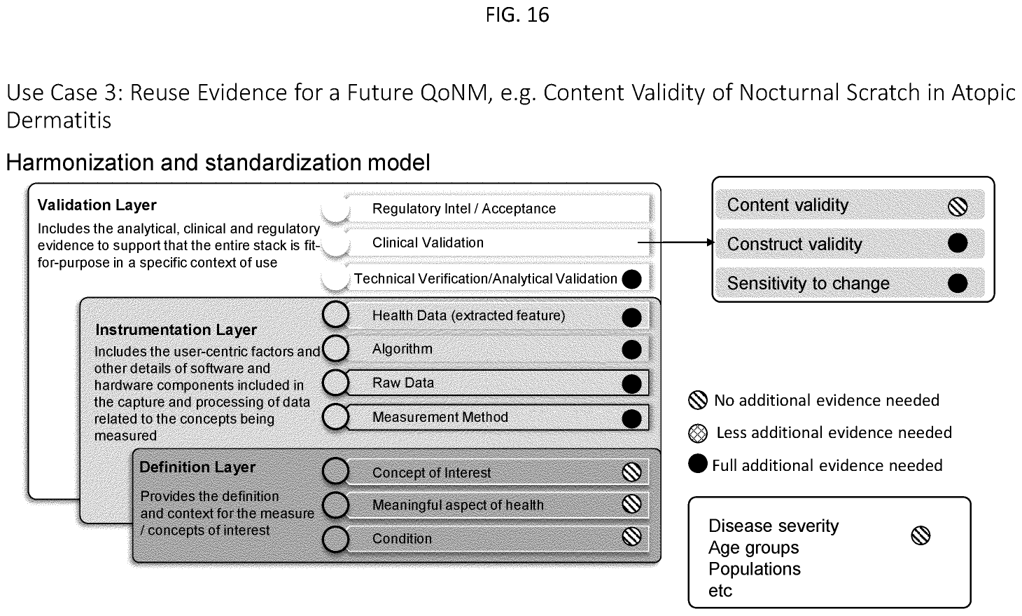

FIG. 16 depicts a third example of acceptability levels of a digital measurement solution. Specifically, this example depicts a reusability of evidence for a future uses. The DMS here is for Nocturnal Scratch and is being reused and applied to Atopic Dermatitis. Here, the validation layer has each of the regulatory intelligence, clinical validation, and technical verification layers as being categorized as needing full additional evidence. This categorization can be due to the construct validity, sensitivity to change, and content validity of each layer of the validation layer. Each layer of the instrumentation layer, such as the health data, algorithm, raw data, and measurement method layers, are all categorized as needing full additional evidence. This is because each of the layers of the instrumentation layer require new data due to validate content of Nocturnal Scratch in Atopic Dermatitis. Each layer of the definition layer, such as the concept of interest, meaningful aspect of health, and condition layers are all categorized as not needing additional evidence, since Nocturnal Scratch is a condition within Atopic Dermatitis.

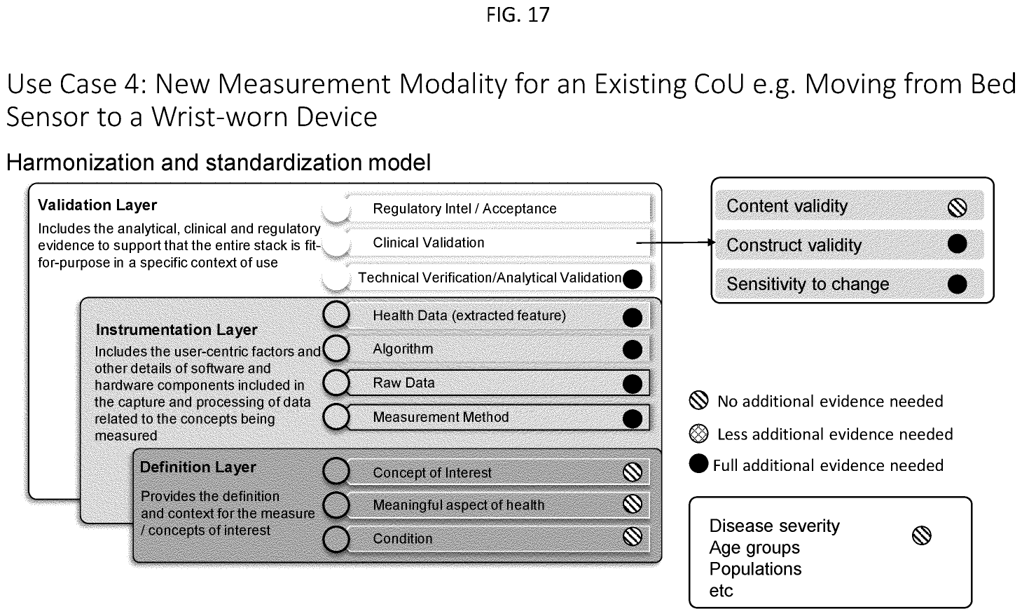

Referring now to FIG. 17, a fourth example of acceptability levels of a digital measurement solution is presented. Specifically, this example depicts a new measurement modality for an existing context of use, for instance moving from a bed sensor to a wrist-worn device. Here, the regulatory intel, clinical validation, and technical verification layers of the validation layer are categorized as needing full additional evidence. The health data, algorithm, raw data, and measurement method layers of the instrumentation layer are also categorized as needing full additional evidence. Here, many of the layers of the DMS are categorized as needing some additional evidence since they are being used for a different purpose rather than transitioning entirely to a new DMS. The concept of interest, meaningful aspect of health, and condition layers of the definition layer are categorized as needing no additional evidence.

### Example 2: Target Solution Profile and Digital Measurement Solutions for Atopic Dermatitis

The measurement stack is divided into nine individual layers. Each component includes unique information that can stand alone and offer additional value combined with the other components. These components have been categorized into three sub-stacks (otherwise referred to as assets): the definition-, instrumentation- and validation sub-stacks. The definition sub-stack describes the condition, meaningful aspect of health, and concepts of interest. Regulatory acceptance of this sub-stack is valuable for pursuing a study and should be aligned early on. The instrumentation sub-stack includes components for collecting, analyzing, and interpreting data. These layers include both specific solutions and generically described classes of solutions. Finally, the validation sub-stack describes the validation studies and regulatory approval to complete the solutions. Although stacks can be built starting from every component, the process often begins with available instrumentation or a medical condition. Atopic dermatitis (AD) is showcased here to walk through the measurement stack and an example target solution profile.

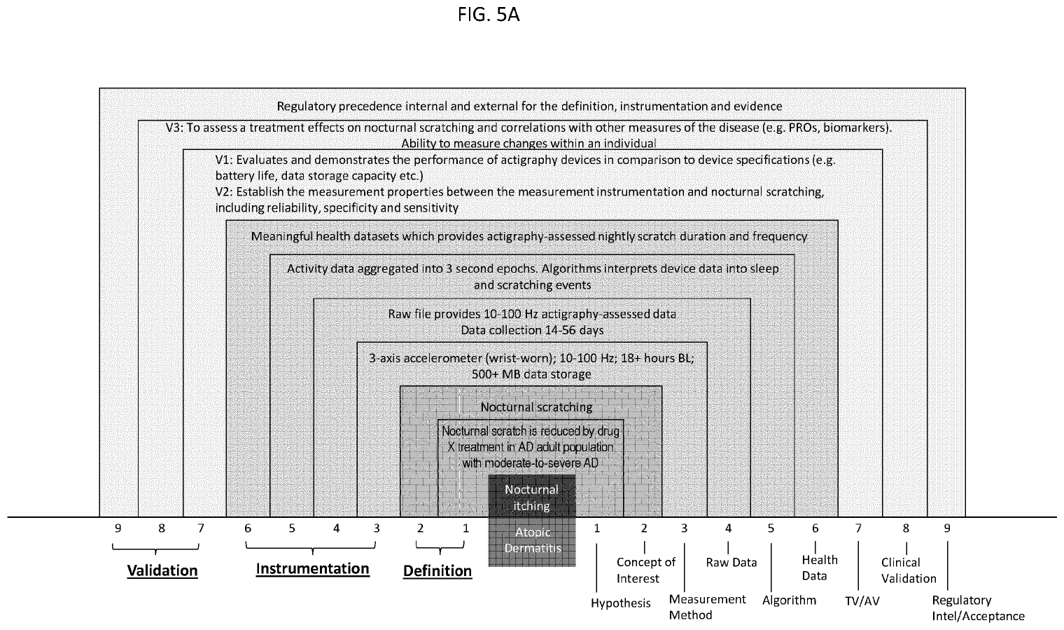

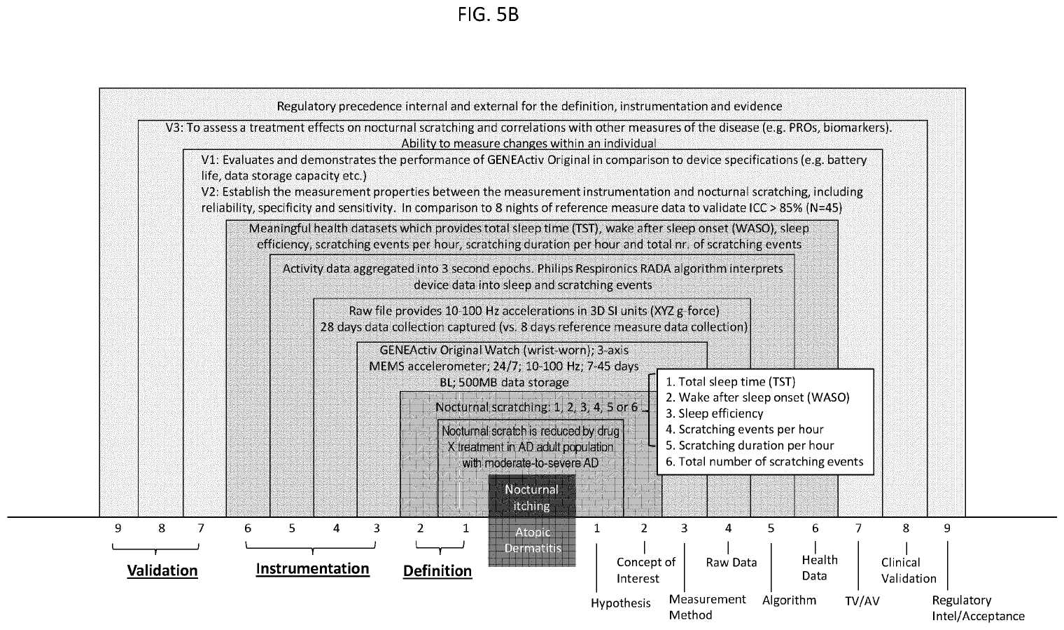

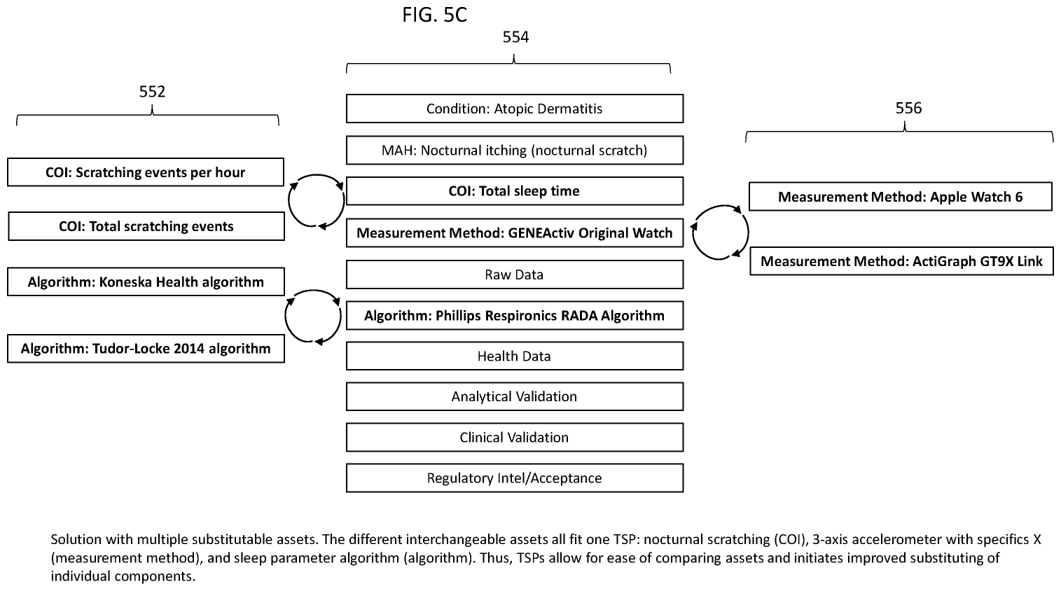

FIG. 5A depicts an example target solution profile for atopic dermatitis. The condition describes the medical condition of the patient population. The meaningful aspect of health (MAH) defines an aspect of this condition which a patient: (1) does not want to become worse, (2) wants to improve, or (3) wants to prevent. Here, the condition is atopic dermatitis and the meaningful aspect of health is nocturnal itching. Referring to the hypothesis component (e.g., component 1 shown in FIG. 5A), it describes the goal of the meaning aspect of health. For example, here, the hypothesis is that nocturnal itching is decreased due to a drug X in an adult population with moderate-to-severe AD.

Component 2 in the measurement stack refers to the concept of interest. Here, the concept of interest (COI) describes how this specific meaningful aspect of health will be measured. For example, if the goal is to measure nocturnal scratching, the concept of interest can be any of a measure of total sleep time, scratching events per hour, or total scratching events. This COI is practically measured using an outcome to measure (OTM). For example, the outcome to measure includes the specific, measurable characteristics of the condition that evaluate the MAH described by the COI. Thus, the outcome to measure reveals whether the treatment of the MAH is beneficial. For example, reduction in scratching events after X weeks. Although not explicitly shown in FIG. 5A, a single condition can have multiple MAHs; one MAH can have multiple COIs; one COI can have multiple outcomes to measure.

Component 3 in the measurement stack refers to the measurement method, which is part of the instrumentation asset. The measurement method includes hardware, software, or firmware solutions. Examples are wearable devices, mobile applications, and sensors. Additionally, complete software solutions can be a measurement method (e.g., speech batteries) as long as the method can reliably measure the OTM. As a specific example, a measurement method is a watch with a 3-axis MEMS accelerometer (10-100 Hz). This device captures accelerations 24/7 for 7 to 45 days (dependent on the set frequency).

Component 4 in the measurement stack refers to the raw data. This component represents the raw datasets which are the outcomes of the measurement method. Generally, raw data does not yet deliver meaningful health data. However, raw data is an individual layer as each of these datasets can be leveraged by multiple algorithms for different purposes. Here, the raw data layer is introduced as, for example, a raw file that provides 10-100 Hz accelerations. This is captured in 3D SI units (XYZ g-force) with 28 days of continuous data collection. In short, this example describes what raw data is captured (accelerations in 3D SI units), its frequency (10-100 Hz), and the amount of data that is captured (28 days).

Component 5 in the measurement stack refers to the algorithm. The algorithm represents a method in transforming raw data into meaningful health datasets. Thus, meaningful health datasets are readily analyzable and interpreted. Algorithms can be integrated into the complete instrumentation, or individual algorithms can be leveraged to transform (individual) raw datasets. For example, Philips Respironics RADA algorithm interprets measurement device data into sleep and scratching events.

Component 6 in the measurement stack refers to the health data. The health data component describes the health dataset generated by the algorithm from the initial raw datasets. Health datasets can be assessed as individual datasets for multiple purposes. Therefore, health datasets are considered a standalone asset and are included in the stack as an individual layer. For example, meaningful health datasets provide total sleep time, scratching events per hour, and the total number of scratching events.

Component 7 in the measurement stack refers to the technical and analytical validations. Further, component 8 in the measurement stack refers to clinical validation. The different validations ensure that digital measures are valid and therefore, can be recognized as eligible for clinical trial approval. Instrumentation outputs are evaluated both in silico and in vitro at the sample level. Specifically, technical validation (referred to as V1 in FIG. 5A) of the digital asset assures that the instrumentation captured the fundamental analog data accordingly. The technical validation verifies whether captured data resulted in the generation of appropriate output data. For example, the technical validation ensures that the instrumentation matches the specifications of the device used to capture the data (e.g., battery life, data storage, and available measure frequencies).

The analytical validation (referred to as V2 in FIG. 5A) evaluates the complete instrumentation, often in vitro. The analytical validation includes validating device specifics, processing algorithms, and reliable health data output. For example, the analytical validation validates the ability to measure, detect and predict physiological or behavioral metrics against an appropriate measurement standard. The specific example in FIG. 5A shows the analytical validation of establishing the measurement properties between the measurement instrumentation and nocturnal scratching, including reliability, specificity, and sensitivity. In comparison to 8 nights of reference measure data to validate ICC>85% (N=45). The ICC validates whether the novel digital instrumentation is better or at least comparable to the reference measure (e.g., infrared observation to monitor nocturnal scratching).

The clinical validation (referred to as V3 in FIG. 5A) evaluates whether the solution identifies, measures, and predicts the meaningful clinical, biological, physical, functional state, or experience in the specific context of use (COU). This is stated by its study design. Clinical validation is the in vivo validation and includes the specific target population and study-specific digital endpoints. The clinical validation allows for a meaningful interpretation of outcomes and evaluates whether the solution is valid to answer clinical questions related to the instrumentation- and definition sub-stack. For example, the clinical validation assesses treatment effects on nocturnal scratching and correlations with other measures of the disease (e.g., PROs, biomarkers) and the ability to measure changes within an individual.

Component 9 of the measurement stack refers to regulatory validation. Here, regulatory qualifications by health authorities are of importance for the acceptance and adoption of digital measures. The regulatory validation assesses all layers of the measurement stack, but predominantly is applied for the definition- and validation components. For example, regulatory precedence internal and external for the definition, instrumentation, and evidence.

As shown in FIG. 5A, the target solution profile for adult Atopic Dermatitis population includes a nocturnal scratching-endpoint based on actigraphy. The TSP represents a generic, solution-agnostic stack. Here, all layers of the instrumentation asset are generically described. The measurement method is a 3-axis wrist-wom accelerometer with a range of specifications. Also, the raw data-, algorithm- and health data layers are generically described, often with a certain scope (e.g., 14-56 days of data collection instead of a specific number of days). Furthermore, the COIs are generically described as nocturnal scratching. Many DMSs, such as the DMS shown in FIG. 5B, are of a class that is represented by this TSP.

FIG. 5B depicts an example digital measurement solution (DMS) for atopic dermatitis. Here, the DMS is populated with a nocturnal scratching endpoint based on actigraphy. The meaningful aspect of health is nocturnal itching, and a total of 6 different concepts of interest have been identified (e.g., total sleep time, wake after sleep onset, sleep efficiency, scratching events per hour, scratching duration per hour, and total number of scratching events). Each of the different concepts of interest can be selected for a particular DMS. Thus, here, the 6 different concepts of interests correspond to 6 different DMSs for atopic dermatitis.

Component 3 (measurement method) in the DMS identifies the particular device that is used for capturing raw data. Specifically, as shown in exemplary FIG. 5B, the GENEActiv Original watch captures the raw data. Furthermore, component 5 (Algorithm) in the DMS identifies the particular algorithm that is used to transform the raw data (component 4) into the health data (component 6). Here, the algorithm in this DMS is the Philips Respironics RADA algorithm that interprets device data into sleep and scratching events.

FIG. 5C depicts the interchangeability of assets of different digital measurement solutions for atopic dermatitis. Here, the interchangeability of assets enables the rapid development of numerous digital measurement solutions incorporating the different assets. For example, the center measurement stack in FIG. 5C refers to the DMS shown in FIG. 5B. The GENEActiv Original Watch is included in component 3 (measurement method). However, other devices such as the Apple Watch (e.g., Apple Watch 6), Fitbit, and ActiGraph (e.g., ActiGraph GT9X Link) can be alternatively included in component 3 in place of the GENEActiv Watch. These additional DMSs are also of the class that is represented by a TSP. Furthermore, the Philips Respironics RADA algorithm can be interchangeable with other types of algorithms, such as the Koneska health algorithm or the Tudor-Locke 2014 algorithm. Furthermore, the particular concept of interest in the measurement stack (e.g., total sleep time) can be interchangeable with other concepts of interests (e.g., scratching events per hour or total scratching events).

### Example 3: Target Solution Profile and Digital Measurement Solutions for Pulmonary Arterial Hypertension

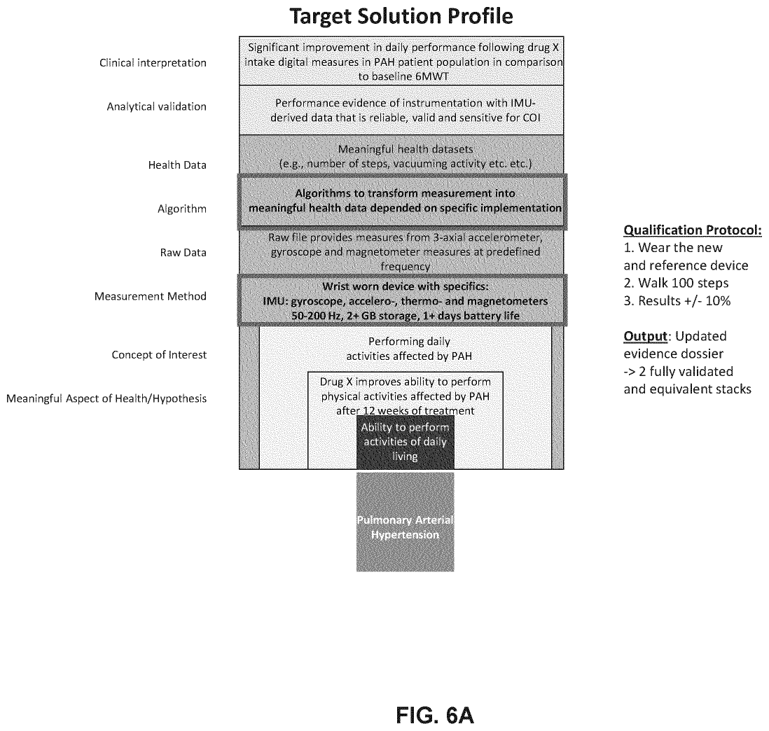

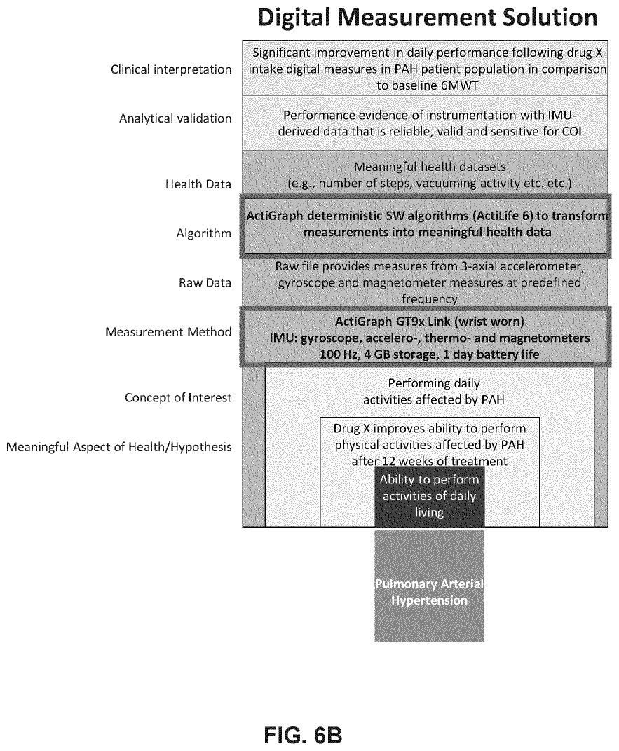

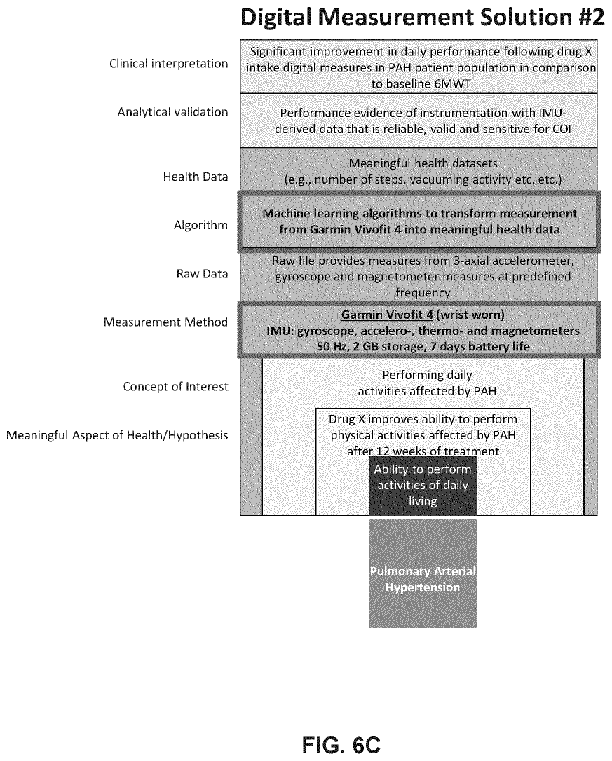

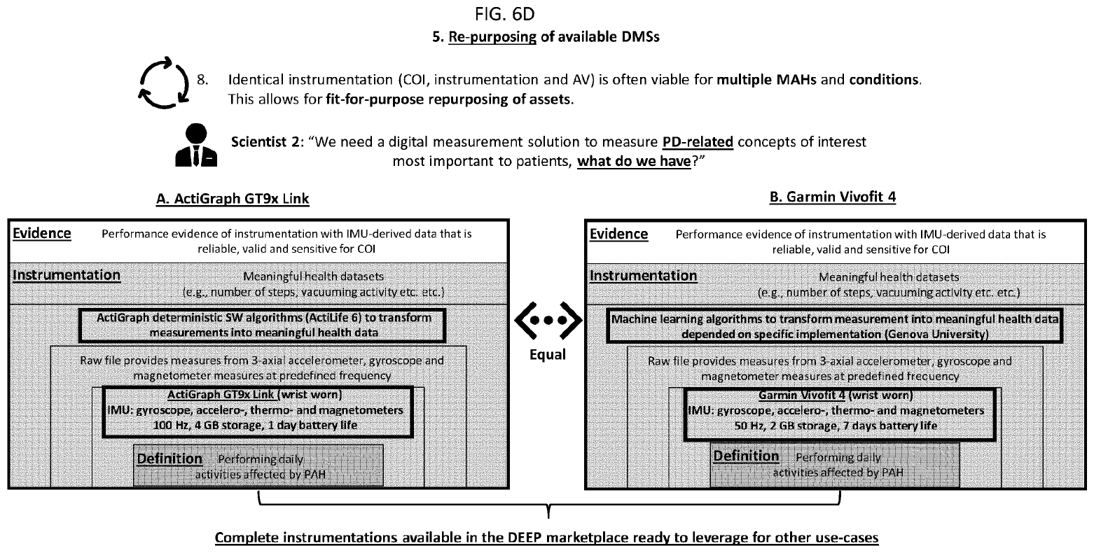

FIG. 6A depicts an example target solution profile for pulmonary arterial hypertension. Here, the meaningful aspect of health is the ability to perform activities of daily living and the corresponding hypothesis is that a particular therapeutic intervention (e.g., drug X) improves ability to perform physical activities. The concept of interest that is to be measured is the ability to perform daily activities while being affected by pulmonary arterial hypertension.

In the instrumentation asset, the measurement method generically identifies a wrist worn device with device specifications. Here, the measurement method is device-technology agnostic and does not identify a particular device nor a particular device-software. The raw data component includes the raw data file that is captured using the measurement method (e.g., wrist worn device). The algorithm component identifies algorithms that transform the raw data file captured using the measurement method into meaningful health data. The health data component includes the meaningful health dataset transformed by the algorithm of the preceding component. As shown in FIG. 6A, examples of meaningful health data for pulmonary arterial hypertension include measures of daily activity such as number of steps, vacuuming activity, and others).

The analytical validation component ensures that the meaningful health dataset is reliable, valid, and sensitive for the concept of interest. The clinical interpretation identifies a significant improvement in daily performance in PAH patients following the drug X intervention.

FIG. 6B depicts a first example digital measurement solution for pulmonary arterial hypertension. Additionally, FIG. 6C depicts a second example digital measurement solution for pulmonary arterial hypertension. Here, each of the digital measurement solution shown in FIGS. 6B and 6C are a common class represented by the target solution profile shown in FIG. 6A.

Referring to the DMS shown in FIG. 6B, the measurement method component specifies a particular wrist worn device (e.g., ActiGraph GT9x Link). Thus, the ActiGraph GT9X Link device captures data which is represented in a raw data file. The algorithm component transforms the raw data file into a meaningful health dataset. As shown in FIG. 6B, ActiGraph deterministic algorithms (e.g., ActiLife 6) is implemented as an algorithm to transform the raw data file into a meaningful health dataset. Altogether, in comparison to the TSP shown in FIG. 6A, the DMS shown in FIG. 6B specifies the particular device of the measurement method component and the particular algorithm of the algorithm component.

Referring to the DMS shown in FIG. 6C, the measurement method component specifies a particular wrist worn device (e.g., Garmin Vivofit 4) that differs from the device specified in FIG. 6B. Thus, the Garmin Vivofit 4 device captures data which is represented in a raw data file. The algorithm component transforms the raw data file into a meaningful health dataset. As shown in FIG. 6C, machine learning algorithms are implemented that transform the measurements captured by the Garmin Vivofit 4 into a meaningful health dataset. Altogether, in comparison to the TSP shown in FIG. 6A, the DMS shown in FIG. 6C specifies the particular device of the measurement method component and the particular algorithm of the algorithm component.

Each digital measurement solution shown in FIGS. 6B and 6C undergoes validation via a qualification protocol to ensure that the respective DMS generates comparable results. An example qualification protocol includes the following:
1) Participants wear the device of the DMS as well as a reference device of a DMS that had previously been successfully validated
2) Participants walk X steps (e.g., 100 steps)
3) Total number of steps measured by the device of the DMS is compared to the total number of steps measured by the reference device. If the difference is less than a threshold number (e.g., 10%), then the device of the DMS is successfully validated.

FIG. 6D depicts the repurposing of at least the instrumentation asset of digital measurement solutions. Here, FIG. 6D shows the target instrumentation profile (e.g., concept of interest component, the instrumentation asset, and analytical validation component) for each DMS shown in FIGS. 6B and 6C. Notably, the target instrumentation profiles of each DMS are interchangeable and viable for other meaningful aspects of health and conditions. Thus, the stack of assets that make up a target instrumentation profile were readily leveraged and incorporated into a different DMS. As a specific example, a digital measurement solution was needed for measuring Parkinson's Disease related concepts of interest. Thus, these target instrumentation profiles that were used for the pulmonary arterial hypertension condition were repurposed for measuring Parkinson's Disease.

### Example 4: Dynamic Digital Document with Smart Parts

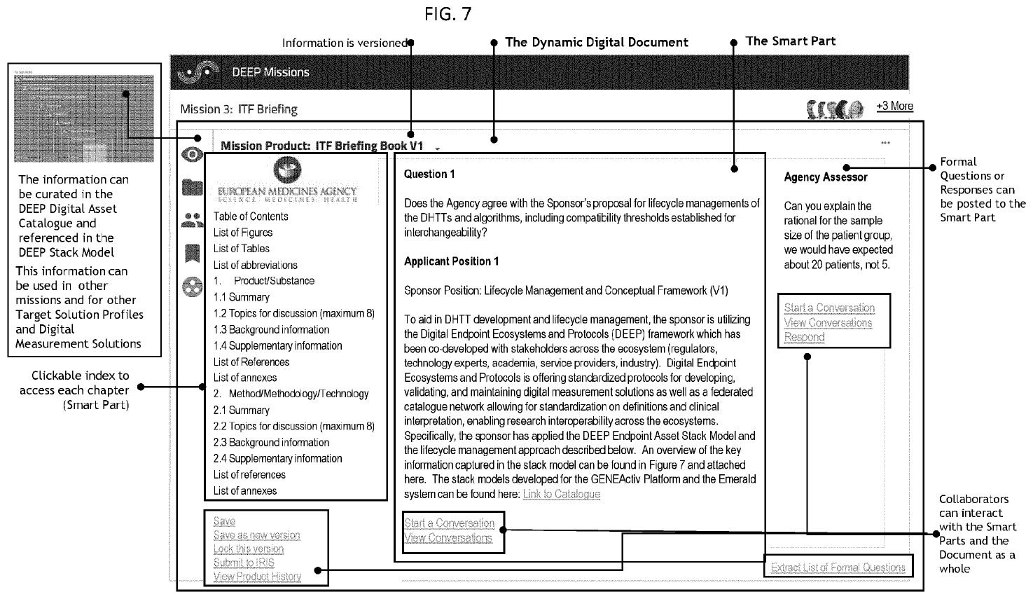

FIG. 7 depicts a dynamic digital document with smart parts. The dynamic digital document is a digital file. The dynamic digital document is located in one or more components of one or more layers of a measurement stack. The dynamic digital document may be representative of a component of a layer of a measurement stack. For instance, the dynamic digital document includes a briefing book of a component of an instrumentation layer. The dynamic digital document includes one or more of text, icons, pictures, and the like. As shown in FIG. 7, the dynamic digital document includes a header. A header includes one or more characters, words, phrases, and the like that may categorize a body of text. The dynamic digital document includes a version history that is representative of a version of the dynamic digital document. The dynamic digital document displays a version of itself at atop, bottom, side, or other location of the dynamic digital document. The dynamic digital document includes an index. An index of the dynamic digital document includes one or more lists of information that are part of the dynamic digital document. A user interacts with an index of the dynamic digital document such as by clicking on a list of a table with a mouse or other user input. The dynamic digital document responds to user input. For example, the dynamic digital document displays a portion of itself that a user may have clicked on in an index table of the dynamic digital document. For instance, a user clicks on a summary tab of the dynamic digital document, to which the dynamic digital document displays a summary.

The dynamic digital document includes one or more smart parts. A smart part of the dynamic digital document may include a modifiable portion of the dynamic digital document. For instance, a smart part includes a body of text may be modifiable by a plurality of users or collaborators. The dynamic digital document include one or more interactive portions. For instance, the dynamic digital document includes a save button, which saves a current version of the dynamic digital document upon receiving user interaction. The dynamic digital document includes a save as new version button, which saves a current version of the dynamic digital document as a new version in a database or other file storage system. The dynamic digital document includes a lock button, which locks a version of the dynamic digital document, preventing modifications from one or more collaborators. The dynamic digital document includes a submission button, which sends a copy, link, and the like, of the dynamic digital document to one or more parties, such as through a network. The dynamic digital document includes a view product history button, which sallow one or more users to see a history of product usage associated with the dynamic digital document.

The dynamic digital document includes a start conversation button. A start conversation button of the dynamic digital document allows two or more users to leave one or more comments, edits, and/or other modification in the dynamic digital document. The dynamic digital document includes a view conversation button, which allows one or more users to view current and/or past conversations of one or more users of the dynamic digital document. The dynamic digital document includes a respond button. A respond button generates a text box for a user to enter text in response to one or more comments of one or more other users. The dynamic digital document further has an extract list of formal questions button. The extract list of formal questions button generates a list of formal questions left by and/or answered by one or more users of the dynamic digital document. The extract list button generates a word, text, or other file of one or more questions and/or answers left by one or more users.

The dynamic digital document and/or other digital documents described throughout this disclosure displays a probability of regulatory success. A probability of regulatory success includes a percentage of approval from one or more regulatory parties. A computing device utilizes a probability model, machine learning model, and/or other machine process to determine a probability of regulatory success. A computing device inputs a variety of data into a probability model and/or machine learning model to determine a probability of regulatory success. Data may include, without limitation, algorithm data, method of measurement data, condition data, patient population data, concept of interest data, analytical validation data, and/or other aspects of one or more layers of a DMS. A computing device is configured to predict, based on a change of patient population in a DMS from adults to pediatrics, that the DMS has an 85% probability of regulatory success. A computing device continually calculates or otherwise updates a probability of regulatory success, for instance as a dynamic digital document or other document is modified and/or updated. A computing device updates and/or calculates a probability of regulatory success based on a data query through one or more databases. For instance, a computing device finds a similar DMS to a proposed DMS and updates and/or calculates a probability of regulatory success based on the similar DMS.

### Example 5: Standardized Solutions for Improved Regulatory Acceptance

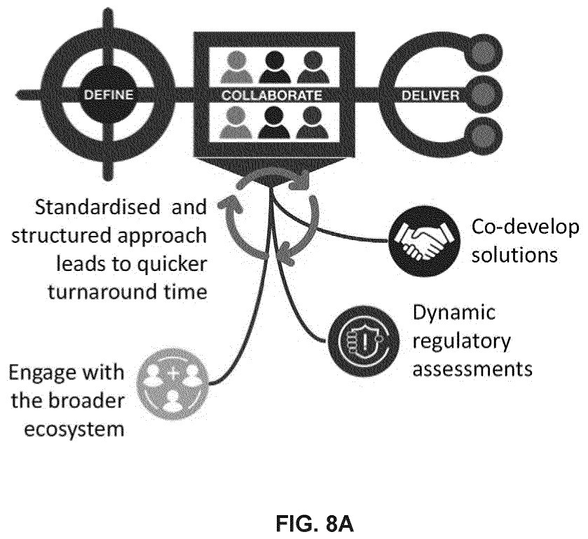

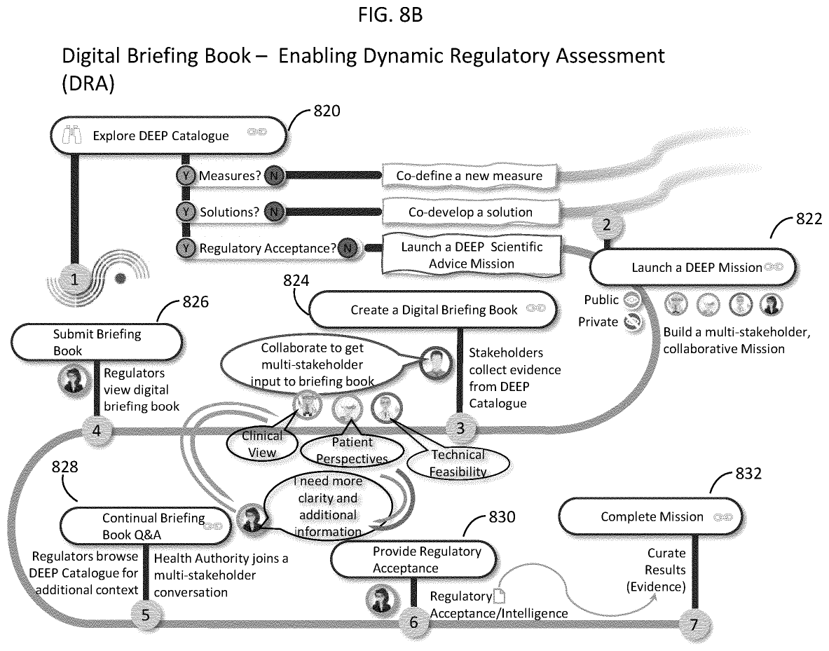

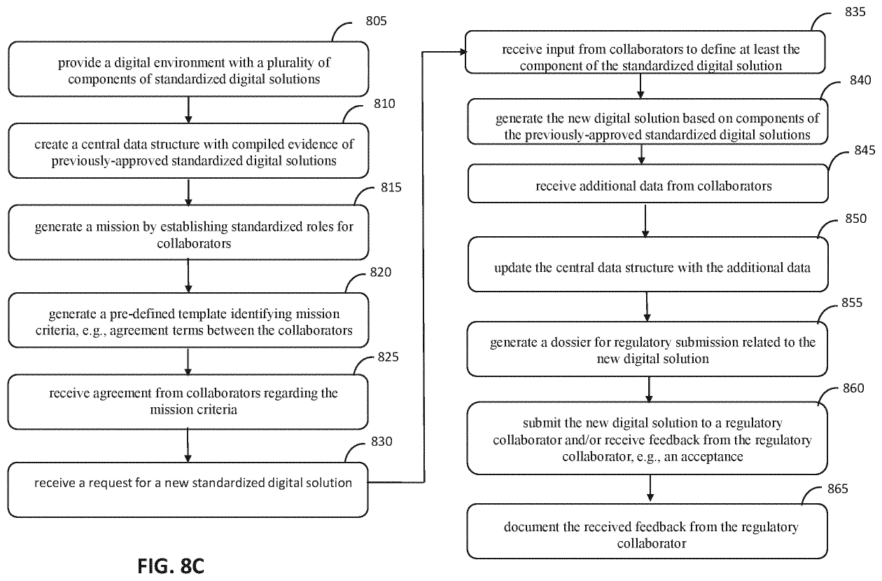

FIG. 8A depicts a high level overview involving collaborative efforts for developing standardized solutions. Here, multiple parties are involved in a collaborative effort for co-developing standardized solutions. These parties engage in a standardized and structured approach, which leads to quicker turnaround and development time. These standardized solutions undergo dynamic regulatory assessments by involving regulators at an early stage during development. Developed solutions are then delivered (e.g., to customers).

FIG. 8B depicts an example flow process involving various parties for enabling dynamic regulatory assessment of standardized solutions. The flow process begins at step 1 involving the Digital Endpoints Ecosystem and Protocols (DEEP) Catalogue. Here, additional digital measures and/or digital solutions are developed and furthermore, these digital solutions can undergo a dynamic regulatory acceptance. Dynamic regulatory acceptance (DRA) involves launching a DEEP mission (step 2), which involves multiple stakeholders that collaborate together on a common Mission. Specifically, at step 3, the stakeholders collaborate to develop a digital briefing book that includes the digital solution. As shown in FIG. 8B, the various stakeholders provide various input into the creation of the briefing book, such as a clinical view, patient perspectives, and/or technical feasibility. At step 4, the briefing book is submitted for regulatory approval such that regulators access the briefing book including the digital solution.

The interaction between regulators and the stakeholders can be as follows: at step 5, the regulator logs in, browses the DEEP catalogue to further understand the digital solution with additional context, and views the public questions as well as the private background materials from both sponsor companies with their private questions. If needed, the regulators can re-engage with the stakeholders to obtain additional clarity and information. The regulator sees that patient and clinician input has already been incorporated. However, the regulator still has questions e.g., it appears important that for severe forms of the disease a comprehensive assessment is made about scratch activity. Thus, regulator asks for additional context regarding patient behavior such as where the patients scratch, how do they scratch, the hours of scratching, etc. The regulators also want to ask patients with less severe disease and understand if their scratch activity is different. The stakeholders (e.g., patient representatives) provide this feedback. Here, patients with severe forms of the disease scratch everywhere and also use both hands and even their feet to scratch. Alternatively, patients with the less severe form of the disease report that usually their itch flares up in one area and they end up scratch just that one spot.

The regulator then wants to ask the patients with a severe form of the disease about the camera solution. In light of their disease, would they tolerate the use of such a solution for periods of time?The patient representative responds that yes, their disease is already burdensome so that they would be willing to do this in order to help find a solution. They do however express concerns about doing this for an extended period of time. The regulator considers all this input and then formulates their response to the questions. In an exemplary response, the regulator agrees that scratch is meaningful and can be used as a key endpoint. Regarding the two solutions envisioned, the regulator sees good applicability for both in different kinds of trials. They both sound plausible, but evidence of their performance to detect scratch activity is required and they also recommend developing evidence to better understand how much change in this measure is going to result in a meaningful benefit to patients. Also the impact on sleep and next day sleepiness should be explored.

Regarding the private questions, the regulator recommends to Pharma company A (severe disease) to consider using both envisioned solutions in their trials. A study could be designed with periods of camera observation as well as wearable device use. It could be studied if the wearable solution could be a suitable surrogate for the more robust video measures. Thus, if the additional value of the video solution can be better understood, better guidance can be provided in the future. An ideal solution could be to use both in studies with severe forms of the disease, balancing scientific value with the burden on patients.

For pharma company B, the regulator foresees that the single wearable device solution could be sufficient to measure these isolated scratching flares. The regulator however recommends that the company also works to understand how well the video and wearable measures correlate and then make an informed choice in their trial design. The regulator recommends the sponsor comes back for more advice when more evidence is available and then discuss specific trial designs again.

Thus, if the regulator sees sufficient evidence, at step 6, the regulator provides regulatory acceptance of the digital solution. At step 7, the collaborative mission involving the multiple stakeholders is completed. The regulatory feedback is curated and connected with the catalogue (MAH, COI and TSP). Stakeholders involved in this regulatory process now have clear direction about next steps. Both solutions have their uses for different purposes and both sponsor companies are already starting to plan for solution development missions. Pharma company A wants to invest in both options, Pharma company B is interested in both, but clearly wants to prioritize the wearable solution development first.

FIG. 8C depicts an example flow process involving various parties for enabling dynamic regulatory assessment of standardized solutions. The flow process begins at step 805 in which various parties can access a digital environment, such as one configured for the DEEP Catalogue. In step 810, central data structure is created with compiled evidence of digital solutions that have received previous approval (in some instances, evidence of one or more aspects of one or more DMS layers). In step 815, a mission is launched, along the lines of the exemplary step 2 in FIG. 8B. In this regard, collaborators, each being in some instances a discrete type of stakeholder, are assigned roles in the mission. Some missions may allow for multiple collaborators with the same role, and some may not. Some missions may designate primary/secondary roles for collaborators, allowing different levels of authority or access to various elements of the mission and related components. In step 820, one or more pre-defined templates may be generated/selected/used, in which various mission identification criteria are identified. For example, the template may reflect an agreement (e.g., a contract) between various stakeholders to the mission, laying out mission criteria. In step 825, agreement is received from the collaborators regarding mission criteria (in some instances, this may involve digitally or otherwise signing an agreement). In step 830, a request for a new digital solution is received, for example from a collaborator, regulator, or other party. In step 835, input is received from one or more collaborators to define one or more components of the standardized digital solution. In step 840, the digital solution is generated based on components of the previously-approved standardized digital solution. In step 845, additional data is received from the collaborators regarding the new digital solution, which is updated to the central data structure in step 850. In some instances, a dossier may be generated in step 855 for submission to a regulatory collaborator (step 860), but in some instances, the dossier may have already be created, e.g., as the central data structure or components thereof. In step 860, the regulatory collaborator may provide feedback, e.g., a regulatory acceptance, and/or a request for more information as described above with reference to FIG. 8B, either of which feedback may be documented digitally, e.g., in connection with the central data structure (step 865).

### Example 6: Example Development of a Standardized Solution Involving Multiple Stakeholders

Assume that Pharmaceutical company A and Pharmaceutical Company B have generated their respective measurement definitions for atopic dermatitis and are interested in measuring number of nighttime scratching events. The remaining question is how to capture these measurements. Pharmaceutical company A would like to develop the right solution for measuring their endpoint. Thus Pharmaceutical company A accesses and searches the DEEP catalogue to identify the solutions that are already in existence.

For example, Pharmaceutical company A types into the asset search box: “scratch”, which results in discovering sensor devices, algorithms and relevant datasets for this use case. Thus, Pharmaceutical A can find the building blocks needed for their solution.

Here, Pharmaceutical company A can create a new mission seeking the services of a Custodian that can assemble a digital solution and maintain it. Pharmaceutical company A will fully fund this work, sets the access rights to Pharmaceutical company A fully owning the solution, but granting an operating license to the Custodian for a period of 3 years, with the Custodian being responsible for maintaining documentation of the solution, including any component upgrades during the licensing period.

The custodian assembles the solution from the components in the catalogue and connects the solution to the measurement definition already established. Pharmaceutical company A can now access the solution they need in their clinical trial.

Additionally, Pharmaceutical company B can now also see the solution being available for licensing from the Custodian (they get a notification that DMS is available for the TSP they are following/subscribed to). The solution performance looks promising and the licensing conditions appears fair. Pharmaceutical company B also decides to license the solution from the Custodian.

Altogether, multiple stakeholders can rapidly adopt solutions through more efficient pathways that are provided through standardized solutions.

### Example 7: Example Decision Tree for Use with Digital Measurement Solutions

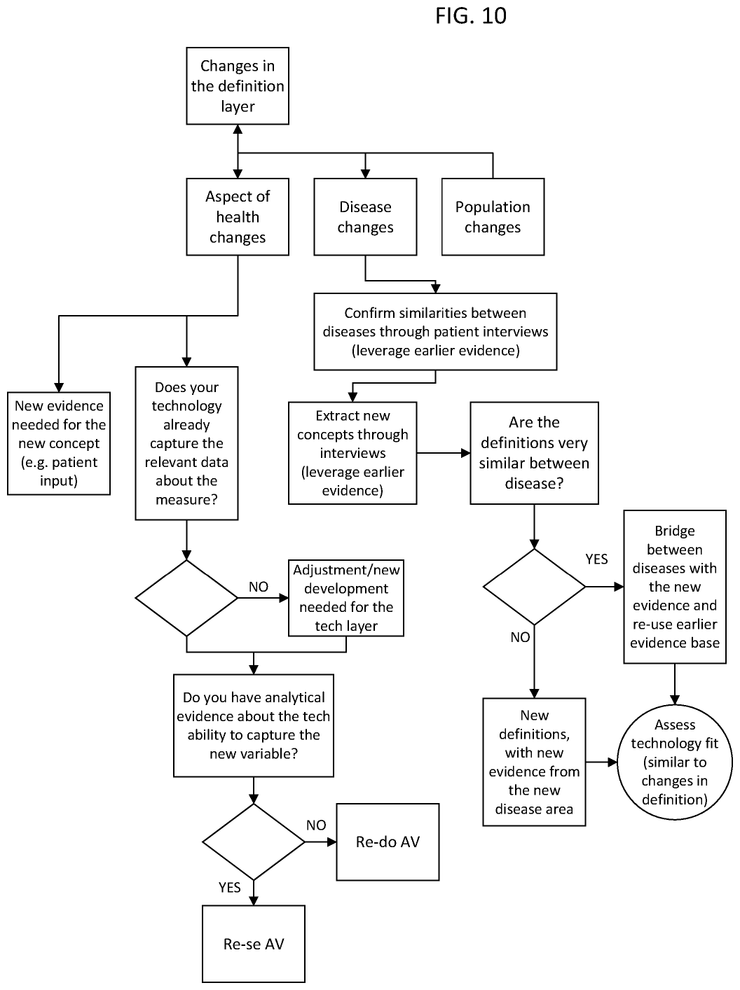

FIG. 10 illustrates an exemplary instance of a decision tree that may be used with any system or process as described throughout this disclosure. As shown in FIG. 10, an aspect of health in a definition layer of a DMS may be changed, updated, and/or modified. If the changes to an aspect of health are not already captured by an algorithm, method of measurement, or other process, the aspect of health needs new evidence for a new concept. New evidence is received through user input, patient input, and the like. Alternatively, if a current technology already captures relevant data about a measurement of the aspect of health, the decision tree moves onto another criteria. A subsequent criteria is included if there is analytical evidence about the technology ability to capture the new variable of the aspect of health change. If there is analytical evidence that a current technology does have an ability to capture a new variable of the aspect of health change, the analytical validation is reused. If there is not analytical evidence about a technological ability to capture a new variable of the aspect of health change, then the analytical validation is reperformed.

Referring back to the decision tree, a change in disease (labeled as “Disease changes”) prompts a criteria comparison. A change in disease includes, as a non-limiting example, spinal muscular atrophy or nocturnal scratch. For instance, a change in a disease of a definition layer of a DMS may be confirmed to be similar to one or more other diseases. Confirmation may come from one or more patient interviews and/or other sources. New concepts of a disease change are extracted through one or more patient interviews. Definitions between diseases are compared for similarity. If the definitions between the diseases are similar, a bridge between the disease is created with new evidence and reused earlier evidence bases. If the definitions between the diseases are not similar, new definitions with new evidence from the new disease area may be generated. An assessment of a technology fit to a new disease is made, which is similar to changes in one or more definitions of a disease.

As further shown in FIG. 10, a population of a DMS may change, which prompts a comparison of the new population with one or more criteria. A population change includes, as a non-limiting example, a population of adults to a population of pediatrics. Similarly to the disease changes, a comparison between previously used populations and a new population is made. If the similarities between the populations are great enough, an analytical validation is reused. If the differences between the populations are great enough, new evidence is generated and a new analytical validation is performed.

### Example 8: Stack Navigation

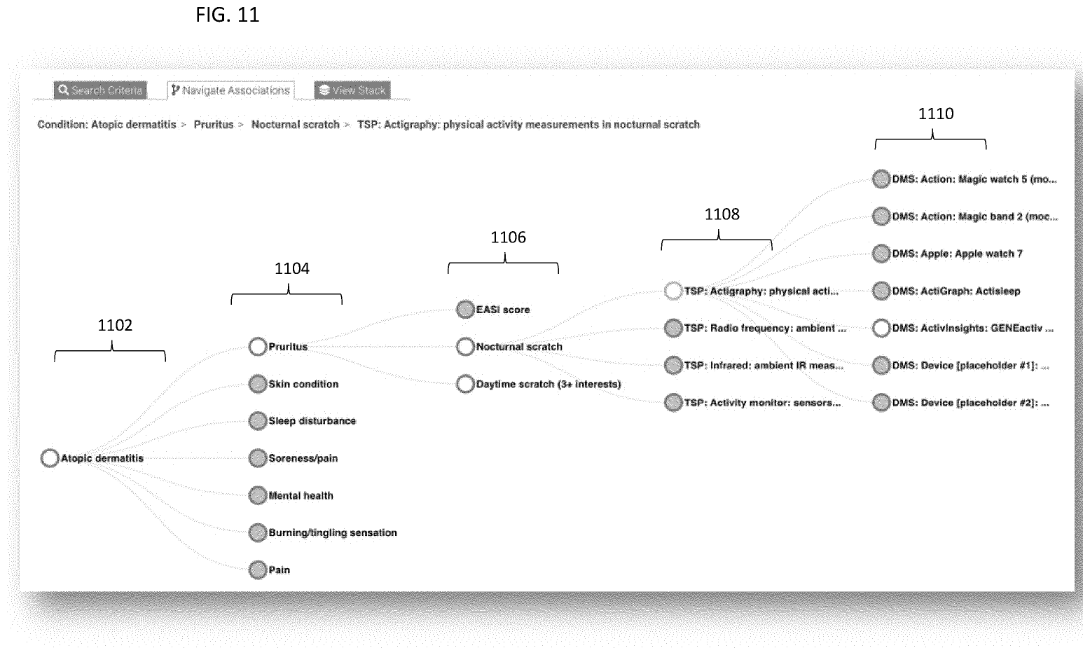

FIG. 11 is an exemplary instance of a user interface for stack navigation. The user interface is displayed on, but not limited to, a laptop, monitor, smartphone, tablet, and the like. The user interface displays a workflow from a left direction of a screen to a right direction of the screen. As shown in FIG. 11, the user interface displays a dendrogram. For instance, at a leftmost portion of a screen, text of a disease is displayed, an example of which is atopic dermatitis. The text of a disease is displayed next to an icon, such as a circle, oval, and the like. The user interface displays a list of symptoms of a disease next to the text of the disease, such as on a right side. For instance, a stack of symptoms next to a stack of circle icons includes, without limitation, pruritus, skin condition, sleep disturbance, soreness/pain, mental health, burning/tingling sensation, pain, and the like. Next to a stack of symptoms, such as a right side of the stack of symptoms, a stack of further symptoms specific to a symptom of the previous stack is displayed next to a stack of circular icons. For instance, a stack of further symptoms includes an EASI score, nocturnal scratch, daytime scratch, and the like. Next to a stack of further symptoms, such as on a right or left side of the stack of further symptoms, a stack of TSP's is displayed. For instance a stack of a TSP for actigraphy, radio frequency, infrared, and/or an activity monitor is displayed. On a right most section of the user interface, one or more DMS's are displayed. For instance, a stack of DMS's are displayed that are specific to a TSP of a previous stack. As a non-limiting example, a TSP of a stack may include actigraphy and a stack of DMS's include a DMS utilizing a Magic watch 5, a magic band 2, an Apple watch 7, an actisleep, a GENEactiv, and/or other measurement instruments and/or methods.

A user selects one or more icons of the user interface, which generates one or more stacks. For instance, a user clicks on a circle icon next to a text of a disease, which generates a stack of symptoms of the disease. A user clicks on a symptom of the stack of symptoms of the disease, which generates further symptoms specific to a first symptom. A user selects a further symptom which generates stacks of one or more TSP's for the symptom. A user selects a TSP which generates a stack of one or more DMS's. Each DMS may be interacted with, which results in a display of a list of ongoing missions, entries from an interest register, and the like.

### Example 9: Cost Savings

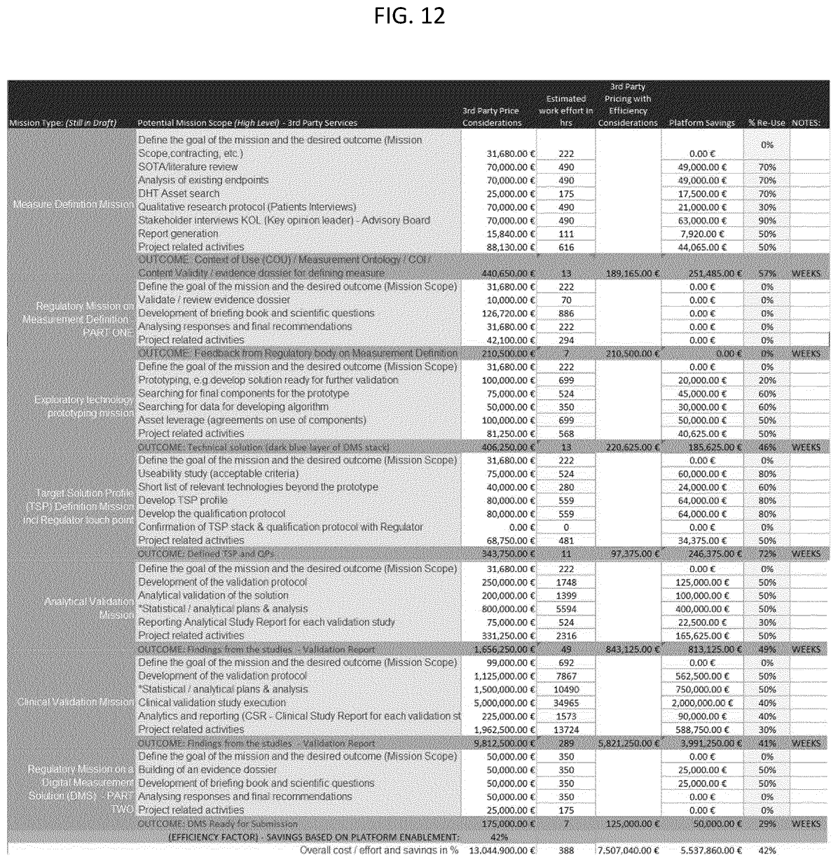

FIG. 12 depicts a screenshot of a user interface showing a chart of cost savings using a DMS. By reusing and/or leveraging previously applied DMS', costs for one or more missions are reduced. For instance, a measurement definition mission includes a literature review, which requires about 490 hours of work effort and about 70,000 Euros in 3^{rd} party price considerations. However, by utilizing a DMS platform and reusing about 70% of previously applied data, about 49,000 Euros of cost of the literature review are saved. Each component of each mission type saves cost through utilizing various percentages of reused data. For instance, an entire measure definition mission typically costs about 440,650 Euros through a 3^{rd} party. However, by reusing data, about 189,165 Euros may be saved, reducing the cost to about 251,485 Euros. Likewise, reusing previously applied data can save costs in regulatory missions, target solution profiles, exploratory technology prototyping missions, analytical validation missions, clinical validation missions, regulator missions on a DMS, and the like. On average, reusing a total of about 42% of previously applied data reduces an overall cost of a combination of each mission from about 13,044,900 Euros by about 7,507,040 Euros to reach a final cost of about 5,537,860 Euros.

### Additional Instances

In various instances, the creation of standardized digital solutions (e.g., TSMPs and DMSs) and/or components of the standardized digital solutions involve a collaborative effort between two or more collaborators within a digital environment. Here, two or more collaborators may embark on a mission generated within the digital environment to provide input on individual components of standardized digital solutions and/or full stacks of standardized digital solutions. For example, within a mission, two or more collaborators may communicate and provide input for defining a measurable concept of interest. As another example, within a mission, two or more collaborators may communicate and provide input for defining a measurement method. Two or more collaborators may communicate and provide input for any component of a digital measurement solution.

In various instances, to improve regulatory review and acceptance of components and/or standardized digital solution, methods disclosed herein involve generating a dossier that includes input from two or more collaborators. Here, the dossier can include input from collaborators for a plurality of components and/or standardized digital solutions. Therefore, the dossier can be provided to a regulator to review and comment on the plurality of components and/or standardized digital solutions.

In various instances, a dossier is structured to include individual elements, where each element is specific for a single component or specific for a single standardized digital solution. Thus, input, communication, and feedback (e.g., from collaborators and the regulator) for a specific component or for a specific standardized digital solution can be compiled and included in a particular element of the dossier. For example, collaborators may provide input to define a component. A regulator may provide questions in response to the input for the component. Here, the collaborator's input can be connected to an element in the dossier specific for the component, and each response from the regulator is then catalogued against that same element in the dossier. Thus, while regulators are presented a complete dossier, the individual elements of that dossier can be deconstructed into individually structured pieces that are specific for individual components. This is beneficial because the regulatory feedback received for a component for one specific use case (e.g., for characterizing a first disease) can be repurposed in other use cases (e.g., for use in standardized digital solutions for characterizing other diseases). For example, if a particular device is used in one case and receives regulatory acceptance, another team can leverage that precedent in a different context of use without having to start from scratch. Thus, this structural approach enables economies of scale across the full digital ecosystem. Altogether, by structuring the dossier with individual elements, the dossier can be delivered to regulators as a single document, but interaction can be centered around these individual elements. For example, collaborators and regulators can communicate within an individual element of the dossier, centered around a specific question or a particular position. Thus, these communications can also be catalogued independently within individual elements. Thus, any advice that is received regarding an asset or component of a standardized solution can be catalogued appropriately. Finally, across the ecosystem, these component parts of regulatory precedent can be repurposed.

Additionally disclosed herein is a method for facilitating regulatory decision-making of at least a component of a standardized digital solution for characterizing a disease, the method comprising: providing a digital environment comprising a plurality of components of standardized digital solutions, wherein the digital environment is accessible by a plurality of collaborators, and wherein two or more collaborators of the plurality have expressed a common interest; generating a mission within the digital environment by establishing standardized roles for two or more collaborators, wherein the mission is created for the two or more collaborators to contribute towards at least the component of the standardized digital solution for seeking regulatory decision-making; receiving input from one or more of the two or more collaborators contributing towards the component of the standardized digital solution for seeking regulatory decision-making; generating a dossier for at least the component of the standardized digital solution, the dossier comprising the received input contributing towards at least the component of the standardized digital solution; and providing access to at least a portion of the dossier within the mission to a regulator collaborator.

In various instances, methods disclosed herein further comprise: receiving feedback from the regulator collaborator specific for the received input contributing towards at least the component of the standardized digital solution; and documenting the received feedback from the regulator collaborator in the dossier. In various instances, the feedback from the regulatory collaborator comprises a regulatory acceptance of at least the component of the standardized digital solution. In various instances, the feedback from the regulatory collaborator comprises one or more questions or requests pertaining to at least the component of the standardized digital solution.

In various instances, methods disclosed herein further comprise: receiving additional input from the two or more collaborators responsive to the feedback from the regulatory collaborator; and documenting the additional input from the two or more collaborators in the dossier accessible within the mission. In various instances, methods disclosed herein further comprise: publishing the dossier outside of the mission for use in one or more subsequent missions. In various instances, establishing roles for the two or more collaborators comprises assigning a primary role to a first of the two or more collaborators and assigning a secondary role to the second of the two or more collaborators. In various instances, the primary role possesses additional rights in comparison to the secondary role. In various instances, establishing roles for the two or more collaborators further comprising assigning one or more additional roles. In various instances, the primary role and the secondary role each possesses additional rights in comparison to the additional role. In various instances, the additional role is assigned to any one of a funder, observer, technology provider, or data partner. In various instances, the rights comprise rights to access or view results developed from the mission. In various instances, the rights comprise rights to use results developed from the mission.

In various instances, prior to generating the mission, selecting the two or more collaborators for inclusion in the mission. In various instances, the common interest expressed by the two or more collaborators is a common interest in contributing towards the component of the standardized digital solution. In various instances, generating the mission comprises generating a private mission accessible only by the two or more collaborators with defined roles. In various instances, generating the mission comprises generating a public mission accessible by additional collaborators. In various instances, the two or more collaborators comprise two, three, four, five, six, seven, eight, nine, ten, eleven, twelve, thirteen, fourteen, fifteen, sixteen, seventeen, eighteen, nineteen, or twenty collaborators.

In various instances, methods disclosed herein further comprise: prior to receiving input from one or more of the two or more collaborators to define at least the component of the standardized digital solution, receiving agreement from each of the two or more collaborators on one or more criteria of the mission. In various instances, the one or more criteria of the mission comprise terms and conditions of an agreement. In various instances, prior to receiving agreement from each of the two or more collaborators, generating a pre-defined template identifying the one or more criteria of the mission; and providing the pre-defined template to each of the two or more collaborators. In various instances, generating the pre-defined template comprises: receiving an input from one of the two or more collaborators for establishing a characteristic of a second role; and incorporating the input from the one of the two or more collaborators into the pre-defined template.

In various instances, the standardized digital solution is one of a digital measurement solution or a target solution profile. In various instances, the target solution profile represents a common class of digital measurement solutions. In various instances, the digital measurement solution comprises: a measurement definition defining one or more concepts of interest relevant to a disease; an instrumentation asset configured to transform a measurement of interest captured according to the measurement definition to a dataset that is informative for characterizing the disease, wherein the instrumentation asset of the digital measurement solution is specific for a device used to capture the measurement of interest. In various instances, the target solution profile comprises: a measurement definition defining one or more concepts of interest relevant to a disease; an instrumentation asset configured to transform data captured according to the measurement definition to a dataset, the instrumentation asset being device technology agnostic and is thereby interchangeable across different target solution profiles. In various instances, a component of the standardized digital solution comprises any of: a measurable concept of interest component; a measurement method component; a raw data component; an algorithm component; a dataset component; a technical validation component; an analytical validation component; a clinical validation component; or a regulatory component.

In various instances, the dossier is further generated for one or more components in addition to the component of the standardized digital solution. In various instances, the dossier is structured to comprise a plurality of elements, wherein each of the plurality of elements of the dossier is specific for a single component selected from the one or more components or the component of the standardized digital solution. In various instances, an element of the dossier specific for a single component stores input contributing towards the single component.

In various instances, methods disclosed herein further comprise receiving feedback from the regulator collaborator specific for one of the one or more components in the dossier; and incorporating the feedback specific for the one component into an element specific for the one component. In various instances, the dossier is further generated for one or more standardized digital solutions in addition to the component of the standardized digital solution. In various instances, the dossier is structured to comprise a plurality of elements, wherein each of the plurality of elements of the dossier is specific for a single standardized digital solution selected from the one or more standardized digital solutions. In various instances, methods disclosed herein further comprise: receiving feedback from the regulator collaborator specific for one of the one or more standardized digital solutions in the dossier; and incorporating the feedback specific for the one standardized digital solution into an element specific for the one standardized digital solution.

In various instances, the digital environment is a collaborative platform built upon a measurement stack model. In various instances, each of the two or more collaborators is a discrete stakeholder. In various instances, the received input comprises one or more of the following types of information: technical feasibility information, patient perspective information, and clinical information. In various instances, the regulatory decision-making comprises regulatory acceptance.

In various instances, methods disclosed herein further comprise: generating a legal agreement based on one or more of the common interest, the regulator collaborator, the regulatory decision, and the established roles of the two or more collaborators, wherein the legal agreement comprises legal terms, the legal terms comprising one or more of: mission rules and collaborator rights; providing, via the digital environment, each of the two or more collaborators with the legal agreement; and providing, via the digital environment, a framework for one or more collaborators to negotiate and edit the legal terms.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

### Additional Instances

Disclosed herein is a method of managing at least a component of a standardized digital solution for characterizing a disease, comprising: obtaining, at a processor, a central data structure, wherein the central data structure comprises compiled evidence of a plurality of components of previously approved standardized digital solutions; receiving, at the processor, a user request for a new standardized digital solution; generating, at the processor, the new standardized digital solution by leveraging one or more components of the previously approved standardized digital solutions in the central data structure; receiving, at the processor, additional data from a user for the new standardized digital solution; updating, at the processor, the central data structure to incorporate the additional data; receiving feedback from a regulatory collaborator specific for a received input contributing towards at least a component of the new standardized digital solution; and documenting the received feedback from the regulatory collaborator, wherein the received feedback comprises a regulatory acceptance of at least the component of the new standardized digital solution.

In various instances, the method further comprises adding, by the processor, the additional data to the new standardized digital solution to create an extended standardized digital solution. In various instances, the processor is further configured to generate a plurality of new standardized digital solutions for different patient populations. In various instances, the different patient populations include one of pediatric, geriatric, or adult population groups.

In various instances, the components of the previously approved standardized digital solutions are included in one or more of a validation layer, an instrumentation layer, or a definition layer of the central data structure. In various instances, the previously approved assets include measurement data of a medical device.

In various instances, generating the new standardized digital solution comprises comparing evidence data of the previously approved assets and generating the new standardized digital solution based on at least an acceptability categorization of the evidence data. In various instances, the acceptability categorization is determined according to one or more of a freshness of the evidence data, a quantity of the evidence data, a patient population of the evidence data, or a combination thereof.

In various instances, the acceptability categorization is determined according to one or more of regulatory intel/acceptance of the evidence data, clinical validation of the evidence data, or technical verification of the evidence data, or a combination thereof. In various instances, the acceptability categorization is determined according to one or more of a content validity of the evidence data, a construct validity of the evidence data, a sensitivity to change of the evidence data, or a combination thereof. In various instances, the acceptability categorization is one of full acceptability, intermediate acceptability, or unacceptability. In various instances, full acceptability corresponds to no additional evidence needed for generating the new standardized digital solution. In various instances, intermediate acceptability corresponds to less additional evidence needed for generating the new standardized digital solution. In various instances, unacceptability corresponds to full additional evidence needed for generating the new standardized digital solution. In various instances, each component of the previously approved standardized digital solution undergoes a form of validation based on the acceptability threshold.

In various instances, at least one component of the instrumentation layer and at least one component of the definition layer are categorized in a full acceptability category. In various instances, at least one component of the definition layer is categorized in a full acceptability category. In various instances, at least one component of the instrumentation layer is categorized in an unacceptability category and at least one component of the definition layer is categorized in a full acceptability category. In various instances, each component of the instrumentation layer and at least two components of the definition layer are categorized as full acceptability.

In various instances, one of a validation layer, an instrumentation layer, a definition layer, or a combination thereof of the previously approved new standardized digital solution undergoes a form of validation based on the acceptability threshold.

In various instances, generating new standardized digital solution comprises generating the new standardized digital solution based on a medical condition. In various instances, generating the new standardized digital solution includes generating the new standardized digital solution based on a diagnosis method.

In various instances, the method further comprises displaying, at a display device, the updated central data structure though a graphical user interface (GUI), wherein the GUI visualizes the additional data added from the user through the new standardized digital solution.

In various instances, the new standardized digital solution includes a diagnostic specific algorithm.

In various instances, the method further comprises: prior to receiving the user request, providing a digital environment comprising a plurality of components of standardized digital solutions, wherein the digital environment is accessible by a plurality of collaborators.

In various instances, the method further comprises: prior to receiving the user request, generating a mission within a digital environment by establishing standardized roles for two or more collaborators. In various instances, the two or more collaborates express a common interest. In various instances, the method further comprises: receiving input from one or more collaborators towards a component of the new standardized digital solution.

In various instances, the method further comprises providing access to at least a portion of a dossier within a mission to a regulator collaborator.

In various instances, the feedback from the regulatory collaborator comprises one or more questions or requests pertaining to at least the component of the new standardized digital solution.

In various instances, the method further comprises: receiving additional input from the two or more collaborators responsive to the feedback from the regulatory collaborator; and documenting the additional input from the two or more collaborators in the dossier accessible within the mission.

In various instances, the method further comprises: publishing the dossier outside of the mission for use in one or more subsequent missions.

In various instances, establishing roles for the two or more collaborators comprises assigning a primary role to a first of the two or more collaborators and assigning a secondary role to the second of the two or more collaborators. In various instances, the primary role possesses additional rights in comparison to the secondary role. In various instances, establishing roles for the two or more collaborators further comprising assigning one or more additional roles. In various instances, the primary role and the secondary role each possesses additional rights in comparison to the additional role. In various instances, the additional role is assigned to any one of a funder, observer, technology provider, or data partner. In various instances, the rights comprise rights to access or view results developed from the mission. In various instances, the rights comprise rights to use results developed from the mission.

In various instances, the method further comprises, prior to generating the mission, selecting the two or more collaborators for inclusion in the mission. In various instances, generating the mission comprises generating a private mission accessible only by the two or more collaborators with defined roles. In various instances, generating the mission comprises generating a public mission accessible by additional collaborators. In various instances, the two or more collaborators comprise two, three, four, five, six, seven, eight, nine, ten, eleven, twelve, thirteen, fourteen, fifteen, sixteen, seventeen, eighteen, nineteen, or twenty collaborators. In various instances, the method further comprises: prior to receiving input from one or more of the two or more collaborators to define at least the component of the standardized digital solution, receiving agreement from each of the two or more collaborators on one or more criteria of the mission.

In various instances, the one or more criteria of the mission comprise terms and conditions of an agreement. In various instances, prior to receiving agreement from each of the two or more collaborators, generating a pre-defined template identifying the one or more criteria of the mission; and providing the pre-defined template to each of the two or more collaborators. In various instances, generating the pre-defined template comprises: receiving an input from one of the two or more collaborators for establishing a characteristic of a second role; and incorporating the input from the one of the two or more collaborators into the pre-defined template.

In various instances, the standardized digital solution is one of a digital measurement solution or a target solution profile. In various instances, the target solution profile represents a common class of digital measurement solutions. In various instances, the digital measurement solution comprises: a measurement definition defining one or more concepts of interest relevant to a disease; an instrumentation asset configured to transform a measurement of interest captured according to the measurement definition to a dataset that is informative for characterizing the disease, wherein the instrumentation asset of the digital measurement solution is specific for a device used to capture the measurement of interest. In various instances, the target solution profile comprises: a measurement definition defining one or more concepts of interest relevant to a disease; an instrumentation asset configured to transform data captured according to the measurement definition to a dataset, the instrumentation asset being device technology agnostic and is thereby interchangeable across different target solution profiles. In various instances, a component of the standardized digital solution comprises any of a measurable concept of interest component; a measurement method component; a raw data component; an algorithm component; a dataset component; a technical validation component; an analytical validation component; a clinical validation component; or a regulatory component.

In various instances, the dossier is further generated for one or more components in addition to the component of the standardized digital solution. In various instances, the dossier is structured to comprise a plurality of elements, wherein each of the plurality of elements of the dossier is specific for a single component selected from the one or more components or the component of the standardized digital solution. In various instances, an element of the dossier specific for a single component stores input contributing towards the single component. In various instances, the dossier is further generated for one or more standardized digital solutions in addition to the component of the standardized digital solution. In various instances, the dossier is structured to comprise a plurality of elements, wherein each of the plurality of elements of the dossier is specific for a single standardized digital solution selected from the one or more standardized digital solutions.

In various instances, the method further comprises: receiving feedback from the regulator collaborator specific for one of the one or more components in the dossier; and incorporating the feedback specific for the one component into an element specific for the one component. In various instances, the method further comprises: receiving feedback from the regulator collaborator specific for one of the one or more standardized digital solutions in the dossier; and incorporating the feedback specific for the one standardized digital solution into an element specific for the one standardized digital solution.

In various instances, the digital environment is a collaborative platform built upon a measurement stack model.

In various instances, each of the two or more collaborators is a discrete stakeholder.

In various instances, the received input comprises one or more of the following types of information: technical feasibility information, patient perspective information, and clinical information.

In various instances, the regulatory decision-making comprises regulatory acceptance.

In various instances, the method further comprises: generating an agreement based on one or more of: the common interest, the regulator collaborator, the regulatory decision, and the established roles of the two or more collaborators, wherein the agreement comprises terms comprising one or more of: mission rules and collaborator rights; providing, via the digital environment, each of the two or more collaborators with the agreement; and providing, via the digital environment, a framework for one or more collaborators to edit the terms.

In various instances, the method further comprises generating a dossier for at least a component of the digital measurement solution for seeking regulatory decision-making.

In various instances, the common interest expressed by the two or more collaborators is a common interest in contributing towards the component of the standardized digital solution.

Additionally discloses herein is an apparatus for managing at least a component of a standardized digital solution for characterizing a disease, comprising: a processor; and a memory communicatively coupled to the processor, wherein the memory contains instructions configuring the processor to: obtain a central data structure, wherein the central data structure comprises compiled evidence of a plurality of components of previously approved standardized digital solutions; receive a user request for a new standardized digital solution; generate a new standardized digital solution by leveraging one or more components of the previously approved standardized digital solutions in the central data structure; receive additional data from a user for the new standardized digital solution; and update the central data structure to incorporate the additional data.

In various instances, the processor is further configured to add the additional data to the new standardized digital solution to create an extended standardized digital solution. In various instances, the processor is further configured to generate a plurality of new standardized digital solutions for different patient populations. In various instances, the components of the previously approved standardized digital solutions are included in one or more of a validation layer, an instrumentation layer, or a definition layer of the central data structure. In various instances, the processor is further configured to display the updated central data structure though a graphical user interface (GUI), wherein the GUI visualizes the additional data added from the user through the new standardized digital solution.

In various instances, the new standardized digital solution is generated based on an acceptability threshold of compiled evidence of the previously approved standardized digital solutions. In various instances, the new standardized digital solution is based on a medical condition. In various instances, the new standardized digital solution is based on a diagnosis method. In various instances, the new standardized digital solution includes a diagnostic specific algorithm.

In various instances, the compiled evidence includes measurement data of a medical device. In various instances, the additional data includes medical data. In various instances, the medical data includes one of heart rate data, blood oxygen data, respiratory data, blood sample data, or a combination thereof.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A method of digitally facilitating regulatory decision-making of at least a component of a standardized digital solution for characterizing a disease, the method comprising:
providing a digital environment comprising a plurality of components of standardized digital solutions, wherein the digital environment is accessible by a plurality of collaborators;
obtaining, at a processor, a central data structure, wherein the central data structure comprises compiled evidence of a plurality of components of one or more previously-approved standardized digital solutions;
receiving, at the processor, a request for a new standardized digital solution;
generating, at the processor, the new standardized digital solution by leveraging one or more components of the plurality of components of the one or more previously-approved standardized digital solutions in the central data structure;
receiving, at the processor, additional data for the new standardized digital solution from a collaborator of the plurality of collaborators;
updating, at the processor, the central data structure to incorporate the additional data;
receiving, at the processor, feedback from a regulatory collaborator, wherein the received feedback comprises a regulatory acceptance of at least a component of the new standardized digital solution; and
digitally documenting the received feedback from the regulatory collaborator.

2. The method of claim 1, wherein the one or more previously-approved standardized digital solutions include measurement data collected via a medical device.

3. The method of claim 1, wherein the processor is further configured to generate a plurality of new standardized digital solutions for different patient populations.

4. The method of claim 1, wherein the components of the one or more previously-approved standardized digital solutions are included in one or more of a validation layer, an instrumentation layer, or a definition layer of the central data structure.

5. The method of claim 1, wherein generating the new standardized digital solution comprises analyzing evidence data of the one or more previously-approved digital solutions and generating the new standardized digital solution based on at least an acceptability categorization of the evidence data.

6. The method of claim 5, wherein the acceptability categorization is determined according to one or more of: a freshness of the evidence data, a quantity of the evidence data, a patient population of the evidence data, regulatory acceptance of the evidence data, clinical validation of the evidence data, or technical verification of the evidence data, a content validity of the evidence data, a construct validity of the evidence data, a sensitivity to change of the evidence data, or a combination thereof.

7. The method of claim 1, wherein generating new standardized digital solution is based on a medical condition or a diagnosis method.

8. The method of claim 1, wherein the new standardized digital solution includes a diagnostic specific algorithm.

9. The method of claim 1, wherein the component of the new standardized digital solution comprises any of:
a measurable concept of interest component;
a measurement method component;
a raw data component;
an algorithm component;
a dataset component;
a technical validation component;
an analytical validation component;
a clinical validation component; or
a regulatory component.

10. The method of any one of claims 1-9, further comprising:
prior to receiving the request, generating a mission within a digital environment by establishing standardized roles for two or more collaborators of the plurality of collaborators,
wherein each of the two or more collaborators is a discrete stakeholder in the mission.

11. The method of claim 10, wherein establishing roles for the two or more collaborators comprises assigning a primary role to a first of the two or more collaborators and assigning a secondary role to the second of the two or more collaborators, and wherein the primary role possesses additional rights in comparison to the secondary role, wherein the rights comprise rights to access, view, and/or use results developed from the mission.

12. The method of claim 10-11, wherein a collaborator of the two or more collaborators is assigned to any one of the following roles: a funder, observer, technology provider, or data partner.

13. The method of any one of claims 10-2, further comprising:
prior to receiving input from one or more of the two or more collaborators to define at least the component of the standardized digital solution, receiving agreement from each of the two or more collaborators on one or more criteria of the mission,
wherein the one or more criteria of the mission comprise terms and conditions of an agreement.

14. The method of claim 13, further comprising: prior to receiving agreement from each of the two or more collaborators, generating a pre-defined template identifying the one or more criteria of the mission; and providing the pre-defined template to each of the two or more collaborators.

15. The method of any of claims 1-14, wherein the new standardized digital solution is a digital measurement solution and wherein the digital measurement solution comprises:
a measurement definition defining one or more concepts of interest relevant to a disease;
an instrumentation asset configured to transform a measurement of interest captured according to the measurement definition to a dataset that is informative for characterizing the disease, wherein the instrumentation asset of the digital measurement solution is specific for a device used to capture the measurement of interest.

16. The method of any of claims 1-15, wherein the new standardized digital solution is a target solution profile representing a common class of digital measurement solutions; and
wherein the target solution profile comprises:
a measurement definition defining one or more concepts of interest relevant to a disease;
an instrumentation asset configured to transform data captured according to the measurement definition to a dataset, the instrumentation asset being device technology agnostic and is thereby interchangeable across different target solution profiles.

17. The method of claim 1, wherein the digital environment is a collaborative platform built upon a measurement stack model.

18. The method of claim 1, wherein the received input comprises one or more of the following types of information: technical feasibility information, patient perspective information, and clinical information.

19. The method of claim 10, wherein the method further comprises:
generating an agreement based on one or more of: the common interest, the regulator collaborator, the regulatory decision, and the established roles of the two or more collaborators, wherein the agreement comprises terms comprising one or more of mission rules and collaborator rights;
providing, via the digital environment, each of the two or more collaborators with the agreement; and
providing, via the digital environment, a framework for one or more collaborators to edit the terms.

20. The method of any one of claims 1-19, further comprising generating a dossier for seeking regulatory decision-making, the dossier being related to at least a component of the new standardized digital solution.
